(19) United States
(12) Patent Application Publication
(10) Pub. No.: US 2022/0137507 A1
(43) Pub. Date: May 5, 2022

(54) COMPOUND, ACID GENERATOR, COMPOSITION, CURED PRODUCT, CURED PRODUCT MANUFACTURING METHOD, AND PATTERN COATING MANUFACTURING METHOD

(71) Applicant: ADEKA CORPORATION, Tokyo (JP)

(72) Inventors: Toshihiko MURAI, Tokyo (JP); Shohei FUJITA, Tokyo (JP); Junya MIYAKE, Tokyo (JP); Izumi MATSUI, Tokyo (JP); Konomi OTSUKA, Tokyo (JP)

(21) Appl. No.: 17/425,308

(22) PCT Filed: Jan. 22, 2020
(86) PCT No.: PCT/JP2020/002102

§ 371 Date: Jul. 22, 2021

(30) Foreign Application Priority Data
Jan. 31, 2019 (JP) 2019-015593

(51) Int. Cl. G03F 7/004; C07C 309/73; C07C 309/65; G03F 7/039; G03F 7/038

(52) U.S. Cl. CPC G03F 7/0045; C07C 309/73; G03F 7/038; G03F 7/039; C07C 309/65

(57) ABSTRACT

A compound represented by formula (A) exhibits excellent balance between acid generation sensitivity and capability of providing a composition with reduced discoloration.

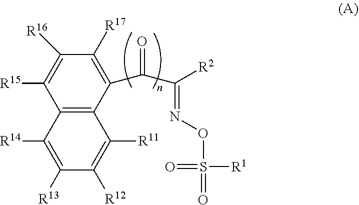

In formula (A), R¹, R¹¹, R¹², R¹³, R¹⁴, R¹⁵, R¹⁶, R¹⁷, and n are as defined in the description. R¹ is preferably an optionally substituted aliphatic hydrocarbon group having 1 to 20 carbon atoms or an optionally substituted aromatic hydrocarbon group having 6 to 20 carbon atoms.

### TECHNICAL FIELD

The present disclosure relates to a compound suited for use as an acid generator.

### BACKGROUND ART

An acid generator is a substance capable of generating an acid upon exposure to energy radiation, such as light, or heating.

Patent literatures 1 and 2 listed below disclose as an acid generator a photo or thermal acid generator comprising a sulfonic acid derivative. The same literatures teach a combined use of the acid generator with a negative resist that reduces in solubility in a developer as a result of chemical bond formation such as polymerization or crosslinking by the action of an acid generated from the acid generator or a positive resist that increases in solubility in a developer as a result of breaking a chemical bond, such as an ester bond or an acetal bond, by the action of an acid. Recommended applications include semiconductors, overcoatings, pigmented or unpigmented coatings, adhesives, and inks.

### CITATION LIST

### Patent Literature

Patent literature 1: JP 2016-169173A
Patent literature 2: EP 3412745(A1)

### SUMMARY OF INVENTION

### Technical Problem

The problem with the above-described acid generators is that a resin composition combining a negative or positive resist resin and the acid generator can undergo discoloration.

In the light of the above problem, it is a main object of the present disclosure to provide a compound having a good balance between acid generation sensitivity and capability of providing a resin composition having reduced discoloration.

### Solution to Problem

As a result of intensive investigations, the inventors have found that a compound having a sulfonic acid derivative structure containing a naphthalene ring and an oxime ester structure functions as an acid generator and is highly effective in preventing discoloration of a composition containing the same and a cured product of the composition. The present invention has thus been completed based on these findings.

The disclosure provides a compound represented by general formula (A) (hereinafter also referred to as the compound A).

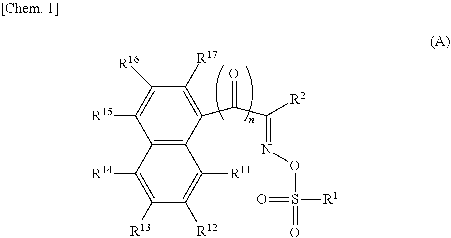

wherein R¹ represents an optionally substituted aliphatic hydrocarbon group having 1 to 20 carbon atoms, an optionally substituted aromatic hydrocarbon group having 6 to 20 carbon atoms, or an optionally substituted heterocyclic group having 2 to 20 carbon atoms, one or more than one methylene group of the aliphatic or aromatic hydrocarbon group or the heterocyclic group being optionally replaced by a divalent group selected from Group I below; R² represents a hydrogen atom, a halogen atom, a nitro group, a cyano group, an optionally substituted aliphatic hydrocarbon group having 1 to 20 carbon atoms, an optionally substituted aromatic hydrocarbon group having 6 to 20 carbon atoms, or an optionally substituted heterocyclic group having 2 to 20 carbon atoms, one or more than one methylene group of the aliphatic or aromatic hydrocarbon group or the heterocyclic group being optionally replaced by a divalent group selected from Group I below; R¹¹, R¹², R¹³, R¹⁴, R¹⁵, R¹⁶, and R¹⁷ each independently represent a hydrogen atom, a halogen-atom, a nitro group, a cyano group, a hydroxy group, a carboxy group, R²⁰, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²²; R²⁰, R²¹, and R²² each independently represent an optionally substituted aliphatic hydrocarbon group having 1 to 20 carbon atoms, an optionally substituted aromatic hydrocarbon group having 6 to 20 carbon atoms, or an optionally substituted heterocyclic group having 2 to 20 carbon atoms, one or more than one methylene group of the aliphatic or aromatic hydrocarbon group or the heterocyclic group being optionally replaced by a divalent group selected from Group I below; R¹¹ and R¹², R¹² and R¹³, R¹³ and R¹⁴, R¹⁴ and R¹⁵, R¹⁵ and R¹⁶, R¹⁶ and R¹⁷, and R²¹ and R²² may be taken together to form a ring; the substituent capable of displacing one or more than one hydrogen atom of the aliphatic or aromatic hydrocarbon group or the heterocyclic group being a halogen atom, a cyano group, a nitro group, a hydroxy group, a thiol group, —COOH, —SO₂H, an isocyanate group, or an alkyl group with 1 to 4 carbon atoms; and n represents 0 or 1. Group I: —O—, —COO—, —OCO—, —CO—, —CS—, —S—, —SO—, —SO₂—, —NR³⁰—, —NR³⁰—CO—, —CO—NR³⁰—, —NR³⁰—COO—, —OCO—NR³⁰—, and —SiR³⁰R³¹—, wherein R³⁰ and R³¹ each independently represent a hydrogen atom or an unsubstituted aliphatic hydrocarbon group having 1 to 20 carbon atoms.

According to the disclosure, the compound A having the above structure achieves a good balance between acid generation sensitivity and effectiveness in providing a composition having reduced discoloration.

In an embodiment of the disclosure, n is preferably 1. The compound A in which n is 1 has high transparency and therefore achieves a better balance between acid generation sensitivity and effectiveness in providing a composition having reduced discoloration.

In an embodiment of the disclosure, R¹ is selected from an optionally substituted C₁₋₂₀ aliphatic hydrocarbon group and an optionally substituted C₆-20 aromatic hydrocarbon group, specifically selected from a C₁₋₁₀ alkyl having one or more than one of its hydrogens replaced by a halogen, a C₆₋₁₅ aryl, and a C₆₋₁₅ aryl having its hydrogen on the ring replaced by an optionally substituted aliphatic hydrocarbon group. The compound A having that structure achieves a better balance between acid generation sensitivity and effectiveness in providing a composition having reduced discoloration.

In an embodiment of the disclosure, R¹¹, R¹², R¹³, R¹⁴, R¹⁵, R¹⁶, and R¹⁷ each independently represent hydrogen, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²². The compound A in which R¹¹, R¹², R¹³, R¹⁴, R¹⁵, R¹⁶, and R¹⁷ have the above structure achieves a better balance between acid generation sensitivity and effectiveness in providing a composition having reduced discoloration.

The disclosure also provides an acid generator containing the compound of formula (A).

According to the disclosure, the acid generator containing the compound A easily provides a composition having reduced discoloration.

The disclosure also provides a composition containing the compound of formula (A) and a resin component.

According to the disclosure, the composition containing the compound A is prevented from discoloration.

In an embodiment of the disclosure, the resin component is an acid-reactive component. The composition which contains the acid-reactive component as a resin component achieves better effects of the compound A in exhibiting acid generation sensitivity and providing a composition with reduced discoloration. The acid-reactive component is preferably an acid-curable component or an acid-decomposable component. The composition which contains the acid-curable component produces a cured product having reduced discoloration. When the composition contains the acid-decomposable component, a non-developed portion of the composition, where no change in solubility in a developer occurs, has reduced discoloration. Furthermore, in the composition containing the acid-decomposable component, deterioration of the acid-decomposable component by oxidation that seems to cause discoloration is prevented, so that the solubility of the acid-decomposable component in a developer easily changes.

The disclosure also provides a cured product of the composition in which the acid-reactive component is the acid-curable component.

According to the disclosure, a cured product with reduced discoloration is obtained easily by using the above-described composition.

The disclosure also provides a method for producing a cured product including a curing step in which the above composition is cured, wherein the acid-reactive component is the acid-curable component.

According to the disclosure, the use of the above composition enables easy production of a cured product with reduced discoloration.

The disclosure also provides a method for forming a patterned coating. The method includes the steps of forming a coating using the above composition, causing a compound in the coating to generate an acid, and developing a part of the coating after the acid generation to form a patterned coating. In the method, the acid-reactive component is the acid-decomposable component.

According to the disclosure, the use of the above composition enables formation of a patterned coating with high dimensional accuracy.

### Advantageous Effects of Invention

The disclosure produces the effect in providing a compound having a good balance between acid generation sensitivity and effectiveness in making a composition having reduced discoloration.

### DESCRIPTION OF EMBODIMENTS

The disclosure relates to a compound, an acid generator, a composition, a cured product of the composition, a method for producing the cured product, and a method for forming a patterned coating.

The compound, acid generator, composition, cured product, cured product production method, and patterned coating formation method according to the disclosure will be described in detail.

### A. Compound

The compound disclosed herein is represented by general formula (A):

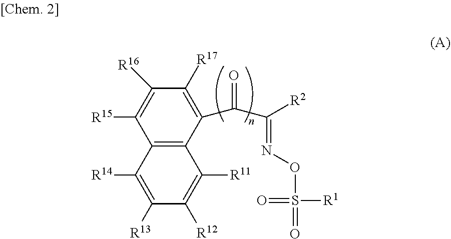

wherein R¹ represents an optionally substituted C₁₋₂₀ aliphatic hydrocarbon group, an optionally substituted C₆₋₂₀ aromatic hydrocarbon group, or an optionally substituted C₂₋₂₀ heterocyclic group, one or more than one methylene group of the aliphatic or aromatic hydrocarbon group or the heterocyclic group being optionally replaced by a divalent group selected from Group I below; R² represents hydrogen, halogen, nitro, cyano, an optionally substituted C₁₋₂₀ aliphatic hydrocarbon group, an optionally substituted C₆₋₂₀ aromatic hydrocarbon group, or an optionally substituted C₂₋₂₀ heterocyclic group, one or more than one methylene group of the aliphatic or aromatic hydrocarbon group or the heterocyclic group being optionally replaced by a divalent group selected from Group I below; R¹¹, R¹², R¹³, R¹⁴, R¹⁵, R¹⁶, and R¹⁷ each independently represent hydrogen, halogen, nitro, cyano, hydroxy, carboxy, R²⁰, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²²; R²⁰, R²¹, and R²² each independently represent an optionally substituted C₁-20 aliphatic hydrocarbon group, an optionally substituted C₆₋₂₀ aromatic hydrocarbon group, or an optionally substituted C₂₋₂₀ heterocyclic group, one or more than one methylene group of the aliphatic or aromatic hydrocarbon group or the heterocyclic group being optionally replaced by a divalent group selected from Group I below; R¹¹ and R¹², R¹² and R¹³, R¹³ and R¹⁴, R¹⁴ and R¹⁵, R¹⁵ and R¹⁶. R¹⁶ and R¹⁷, and R²¹ and R²² may be taken together to form a ring; the substituent capable of replacing one or more than one hydrogen of the aliphatic or aromatic hydrocarbon group or the heterocyclic group being selected from halogen, cyano, nitro, hydroxy, thiol, —COOH, —SO₂H, isocyanate, and a C₁₋₄ alkyl; and n is 0 or 1. Group I: —O—, —COO—, —OCO—, —CO—, —CS—, —S—, —SO—, —SO₂—, —NR³⁰—, —NR³⁰—CO—, —CO—NR³⁰—, —NR³⁰—COO—, —OCO—NR³⁰—, and —SiR³⁰R³¹—, wherein R³⁰ and R³¹ each independently represent hydrogen or an optionally substituted C₁₋₂₀ aliphatic hydrocarbon group.

The compound of formula (A), i.e., the compound A exhibits a good balance between acid generation sensitivity and capability of providing a composition that undergoes reduced discoloration.

Although it is unclear why the compound A has a good balance between acid generation sensitivity and prevention of discoloration in the resulting composition, the inventors believe as follows.

The compound A achieves high acid generation efficiency owing to its sulfonic acid derivative structure. In addition, because the compound A after acid generation offers a structure complementary to the radical that causes deterioration of a resin, the resin is prevented from oxidative deterioration. It seems that, because of the above mechanism, the compound A is excellent in balance between acid generation sensitivity and prevention of discoloration in the resulting composition.

The compound A of the present invention will be illustrated in detail.

The compound A is represented by general formula (A).

The aliphatic hydrocarbon group as represented by R¹, R², R²⁰, R²¹, and R²² in formula (A) and R³⁰ and R³¹ in Group I (hereinafter collectively referred to as R¹, etc.) has 1 to 20 carbon atoms and is substituted or unsubstituted. Any aliphatic hydrocarbon group except hydrocarbon groups containing an aromatic hydrocarbon ring or a heterocyclic ring may be used, including a C₁₋₂₀ alkyl, a C₂₋₂₀ alkenyl, a C₃-20 cycloalkyl, and a C₄-20 cycloalkylalkyl, with one or more than one hydrogen thereof optionally replaced by a substituent, which will be described later.

The C₁₋₂₀ alkyl as represented by R¹, etc. may be straight or branched. Examples of the straight alkyls include methyl, ethyl, propyl, butyl, iso-amyl, tert-amyl, hexyl, heptyl, and octyl. Examples of the branched alkyl include iso-propyl, sec-butyl, tert-butyl, iso-butyl, iso-pentyl, tert-pentyl, 2-hexyl, 3-hexyl, 2-heptyl, 3-heptyl, iso-heptyl, tert-heptyl, iso-octyl, tert-octyl, 2-ethylhexyl, nonyl, isononyl, decyl, dodecyl, tridecyl, tetradecyl, pentadecyl, hexadecyl, heptadecyl, and octadecyl.

The C₂₋₂₀ alkenyl as represented by R¹, etc. is preferably straight, which may be either a terminal alkenyl (having an unsaturated bond at the end of the carbon chain) or an internal alkenyl (having an unsaturated bond inside the carbon chain, not on the ends). Examples of the C₂₋₂₀ terminal alkenyl include vinyl, allyl, 2-methyl-2-propenyl, 3-butenyl, 4-pentenyl, and 5-hexenyl. Examples of the C₂₋₂₀ internal alkenyl include 2-butenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, and 4,8,12-tetradecatrienylallyl.

The C₃₋₂₀ cycloalkyl as represented by R¹, etc. may be a C₃₋₂₀ saturated monocyclic alkyl or a C₃₋₂₀ saturated polycyclic alkyl, with one or more of hydrogens on their ring optionally replaced by one or more alkyls. Examples of the C₃₋₂₀ saturated monocyclic alkyl include cyclopropyl, cyclobutyl, cyclopentyl, cyclohexyl, cycloheptyl, cyclooctyl, cyclononyl, and cyclodecyl. Examples of the C₃₋₂₀ saturated polycyclic alkyl include adamantyl, decahydronaphthyl, octahydropentalenyl, and bicyclo[1.1.1]pentanyl. Examples of the alkyl that can replace the hydrogen on the ring of the saturated mono- or polycyclic alkyl include those recited above as examples of the C₁₋₂₀ alkyl represented by R¹, etc. The saturated polycyclic alkyl having one or more hydrogens on its ring replaced by the alkyl is exemplified by bornyl.

The term “C₄₋₂₀ cycloalkylalkyl” as represented by R¹, etc. refers to a group having 4 to 20 carbon atoms and composed of an alkyl the hydrogen of which is replaced by a monocyclic or polycyclic cycloalkyl. Examples of the C₄₋₂₀ cycloalkylalkyl having a monocyclic cycloalkyl include cyclopropylmethyl, 2-cyclobutylethyl, 3-cyclopentylpropyl, 4-cyclohexylbutyl, cycloheptylmethyl, cyclooctylmethyl, 2-cyclononylethyl, and 2-cyclodecylethyl. Examples of the C₄₋₂₀ cycloalkylalkyl having a polycyclic cycloalkyl include 3,3-adamantylpropyl and decahydronaphthylpropyl.

The number of carbon atoms of a group as referred to in the description is the total number of carbon atoms of the group inclusive of that of the substituent(s) if any. For instance, when a C₁₋₂₀ alkyl group has its hydrogen substituted, the number 1 to 20 refers to the number of carbon atoms of the substituted alkyl (not the unsubstituted alkyl).

The number of carbon atoms of a group of which the methylene is replaced with a divalent group is identical to that of the group before the replacement of the methylene. For instance, a C₁₋₂₀ alkyl with its methylene replaced with a different divalent group should have 1 to 20 carbon atoms.

The aromatic hydrocarbon group as represented by R¹, R², R²⁰, R²¹, and R²² contains 6 to 20 carbon atoms and optionally has a substituent. Any aromatic hydrocarbon group that contains an aromatic hydrocarbon ring and no heterocyclic ring may be used. Examples of the aromatic hydrocarbon group include a C₆₋₂₀ aryl, a C₇₋₂₀ arylalkyl, and an unsaturated aliphatic hydrocarbon group substituted with an aryl, one or more of the hydrogen atoms possessed by these groups being optionally substituted by a substituent that will be described later.

The C₆₋₂₀ aryl as represented by R¹, R², R²⁰, R²¹, and R²² has aromaticity. The aryl may have a monocyclic structure or a fused ring structure. The aryl may be composed of a monocyclic aryl linked to a monocyclic aryl, a monocyclic aryl linked to a fused aryl, or a fused aryl linked to a fused aryl. The linking group that connects the two aryls may be a single bond, carbonyl, and so on. Examples of the aryl having a single-bonded, monocyclic structure include phenyl, biphenylyl, and benzophenonyl. Examples of the aryl having a fused ring structure include naphthyl, anthracenyl, phenanthryl, and pyrenyl. The hydrogen of the aryl may be replaced with a substituted or unsubstituted aliphatic hydrocarbon group. The aryl as R¹ or R² preferably has 6 to 15 carbon atoms. The aryl as R¹, R², R²⁰, R²¹, and R²² is preferably monocyclic aryl, more preferably phenyl.

Examples of the substituted or unsubstituted aliphatic hydrocarbon group that can replace the hydrogen of the aryl include those recited above as examples of the substituted or unsubstituted C₁₋₂₀ aliphatic hydrocarbon group as represented by R¹, etc. The aliphatic hydrocarbon substituents are preferably exemplified by a C₁₋₄ alkyl and a C₁₋₄ alkyl having all the hydrogens replaced by halogens.

The C₇₋₂₀ arylalkyl as represented by R¹, R², R²⁰, R²¹, and R²² is any one of the above-described alkyls having one or more than one hydrogen thereof replaced by the above-described aryl. Examples of the C₇₋₂₀ arylalkyl include benzyl, fluorenyl, indenyl, 9-fluorenylmethyl, α-methylbenzyl, α,α-dimethylbenzyl, phenylethyl, and naphthylpropyl, with the hydrogen on their ring being optionally replaced by a substituted or unsubstituted aliphatic hydrocarbon group. Examples of the substituted or unsubstituted aliphatic hydrocarbon group that can replace the hydrogen of the alkyl of the arylalkyl and the hydrogen of the arylalkyl include those recited above as examples of the C₁₋₂₀ substituted or unsubstituted aliphatic hydrocarbon group as R¹, etc.

The heterocyclic group as represented by R¹, R², R²⁰, R²¹, and R²² contains 2 to 20 carbon atoms and optionally has a substituent. Examples of the heterocyclic group include groups derived from hetero rings, such as epoxy, oxetanyl, pyridyl, quinolyl, thiazolyl, tetrahydrofuranyl, dioxoranyl, tetrahydropyranyl, morpholylfuranyl, thienyl, methylthienyl, hexylthienyl, benzothienyl, pyrrolyl, pyrrolidinyl, imidazolyl, imidazolidinyl, imidazolinyl, pyrazolyl, pyrazolidinyl, piperidinyl, and piperazinyl; and aliphatic hydrocarbon groups having one or more than one of their hydrogens replaced with a hetero ring; one or more of the hydrogens of these heterocyclic groups being optionally replaced by the substituent that will be described later. The aliphatic hydrocarbon groups are exemplified by the same examples as recited for the substituted or unsubstituted C₁₋₂₀ aliphatic hydrocarbon groups as R¹, etc. Throughout the description, the numbers 2 to 20 in the C₂₋₂₀ heterocyclic group refer to the numbers of carbon atoms not of the hetero ring but of the heterocyclic group.

The halogen as represented by R², R¹¹, R¹², R¹³, R¹⁴, R¹⁵, R¹⁶, and R¹⁷ (hereinafter collectively referred to as R², etc.) is exemplified by fluorine, chlorine, bromine, and iodine.

When the aliphatic or aromatic hydrocarbon group or heterocyclic group has two or more of their methylene groups replaced with divalent groups selected from the above Group I, the two or more divalent groups may be either the same or different. Note that the two or more divalent groups should not adjoin each other.

The aliphatic hydrocarbon group having one or more of its methylene groups replaced by a divalent group selected from the above Group I is exemplified by a bornyl group having its methylene group replaced by —CO—, namely 10-camphoryl, and so forth.

The ring formed by R¹¹ taken together with R¹², R¹² taken together with R¹³, R¹³ taken together with R¹⁴, R¹⁴ taken together with R¹⁵, R¹⁵ taken together with R¹⁶, R¹⁶ taken together with R¹⁷, or R²¹ taken together with R²² may be a 5- to 7-membered monocyclic ring or a fused ring. Examples of the monocyclic ring include monocyclic cycloalkanes, such as cyclopentane, cyclohexane, and cyclopentene; monocyclic aromatic rings, such as benzene; and monocyclic hetero rings, such as pyrrolidine, pyrrole, piperazine, morpholine, thiomorpholine, tetrahydropyridine, lactones, and lactams. Examples of the fused ring include naphthalene and anthracene.

Examples of the substituent that can replace the hydrogen of the aliphatic or aromatic hydrocarbon groups and heterocyclic groups described with reference to general formula (1), which may have one or more methylenes replaced by a divalent group selected from the above Group I, include halogen, cyano, nitro, hydroxy, thiol, —COOH, —SO₂H, isocyanate, and C₁₋₄ alkyl. Preferred of them are halogen and C₁₋₄ alkyl. The C₁₋₄ alkyl may have one or more of its hydrogens replaced by halogen. Examples of the halogen and the C₁₋₄ alkyl include those recited above for use in R¹, R², and so on.

Examples of the halogen that can be used as a substituent include those listed for use in R², etc.

Examples of the aliphatic hydrocarbon group having one or more of its hydrogens replaced by halogen, namely haloalkyl, include trifluoromethyl, pentafluoroethyl, 2-chloroethyl, 2-bromoethyl, heptafluoropropyl, 3-bromopropyl, nonafluorobutyl, tridecafluorohexyl, heptadecafluorooctyl, 2,2,2-trifluoroethyl, 1,1-difluoroethyl, 1,1-difluoropropyl, 1,1,2,2-tetrafluoropropyl, 3,3,3-trifluoropropyl, 2,2,3,3,3-pentafluoropropyl, norbornyl-1,1-difluoroethyl, norbornyltetrafluoroethyl, adamantane-1,1,2,2-tetrafluoropropyl, and bicyclo[2.2.1]heptane-tetrafluoromethyl.

R¹ in formula (A) is preferably the C₁₋₂₀, substituted or unsubstituted aliphatic hydrocarbon group or C₆₋₂₀ substituted or unsubstituted aromatic hydrocarbon group.

The C₁₋₂₀, substituted or unsubstituted aliphatic hydrocarbon group as by R¹ is preferably a C₁₋₁₀, substituted or unsubstituted alkyl. The C₁₋₁₀ alkyl preferably has at least one of its hydrogens replaced by halogen. More preferably, the C₁₋₁₀ alkyl is a C₁_s alkyl having all of its hydrogens replaced by halogen atoms, i.e., a C₁₋₅ perhaloalkyl, even more preferably a C₁₋₅ alkyl having all of its hydrogens replaced by fluorine, i.e., a C₁₋₅ perfluoroalkyl.

The C₆₋₂₀, substituted or unsubstituted aromatic hydrocarbon group as R¹ is preferably a C₆₋₁₅ aryl or a C₆₋₁₅ aryl having one or more of its hydrogens replaced by an optionally substituted aliphatic hydrocarbon group, more preferably a C₇₋₁₀ phenyl having one or more of its hydrogens on the ring replaced by a C₁_4, substituted or unsubstituted alkyl, even more preferably a C₇₋₁₀ group derived from phenyl by replacing one hydrogen on the ring by an unsubstituted C₁₋₄ alkyl, e.g., tolyl. When R¹ is the C₇₋₁₀ phenyl having one of its hydrogens on the ring replaced by an unsubstituted C₁₋₄ alkyl, the unsubstituted C₁₋₄ alkyl is preferably at the para-position with respect to the bond to the sulfur atom. The compound A in which R¹ has the preferred structure exhibits better balance between acid generation sensitivity and effectiveness in providing a composition with reduced discoloration.

R² in formula (A) is preferably a C₁₋₂₀, substituted or unsubstituted aliphatic hydrocarbon group or a C₆₋₂₀, substituted or unsubstituted aromatic hydrocarbon group. The C₁₋₂₀, substituted or unsubstituted aliphatic hydrocarbon group as R² is preferably a C₁₋₁₀ alkyl, more preferably a C₁₋₄ alkyl, even more preferably a C₁₋₂ alkyl. The C₆₋₂₀, substituted or unsubstituted aromatic hydrocarbon group as R² is preferably a C₆₋₁₅ aryl or the aryl having one or more of its hydrogens on the ring replaced by an optionally substituted aliphatic hydrocarbon group, more preferably phenyl or naphthyl, even more preferably phenyl.

The compound A in which R² has the above preferred structure exhibits better balance between acid generation sensitivity and effectiveness in providing a composition having reduced discoloration.

R¹¹, R¹², R¹³, R¹⁴, R¹⁵, R¹⁶, and R¹⁷ in formula (A) each preferably independently represent hydrogen, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²². The compound A in which R¹¹ to R¹⁷ have this preferred structure achieves better balance between acid generation sensitivity and effectiveness in providing a composition having reduced discoloration.

In the disclosure, R²⁰, R²¹, and R²² are each preferably a C₁₋₂₀, substituted or unsubstituted aliphatic hydrocarbon group, more preferably an unsubstituted C₁₋₁₀ aliphatic hydrocarbon group, even more preferably an unsubstituted C₁₋₅ aliphatic hydrocarbon group, still even more preferably an unsubstituted C₁₋₅ alkyl. The compound A in which R²⁰, R²¹, and R²² have this preferred structure achieves better balance between acid generation sensitivity and effectiveness in providing a composition having reduced discoloration.

In the disclosure, R¹¹, R¹², R¹³, R¹⁴, R¹⁶, and R¹⁷ are each preferably a hydrogen atom. The compound A having such a structure achieves better balance between acid generation sensitivity and effectiveness in providing a composition having reduced discoloration.

In the disclosure, R¹⁵ is preferably hydrogen, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²², more preferably hydrogen or OR²⁰. When R¹⁵ is OR²⁰, the R²⁰ is preferably an unsubstituted C₁₋₅ aliphatic hydrocarbon group, more preferably an unsubstituted C₁₋₅ alkyl. The compound A in which R¹⁵ has this structure exhibits better balance between acid generation sensitivity and effectiveness in providing a composition having reduced discoloration. When R¹⁵ is hydrogen or OR²⁰, R¹¹, R¹², R¹³, R¹⁴, R¹⁶, and R¹⁷ are each preferably hydrogen.

The symbol n in formula (A) is preferably 1. The compound A in which n is 1 is excellent in transparency and, as a consequence, exhibits better balance between acid generation sensitivity and effectiveness in providing a composition having reduced discoloration.

Specific examples of the compound A include the following compounds numbered 1 through 164.

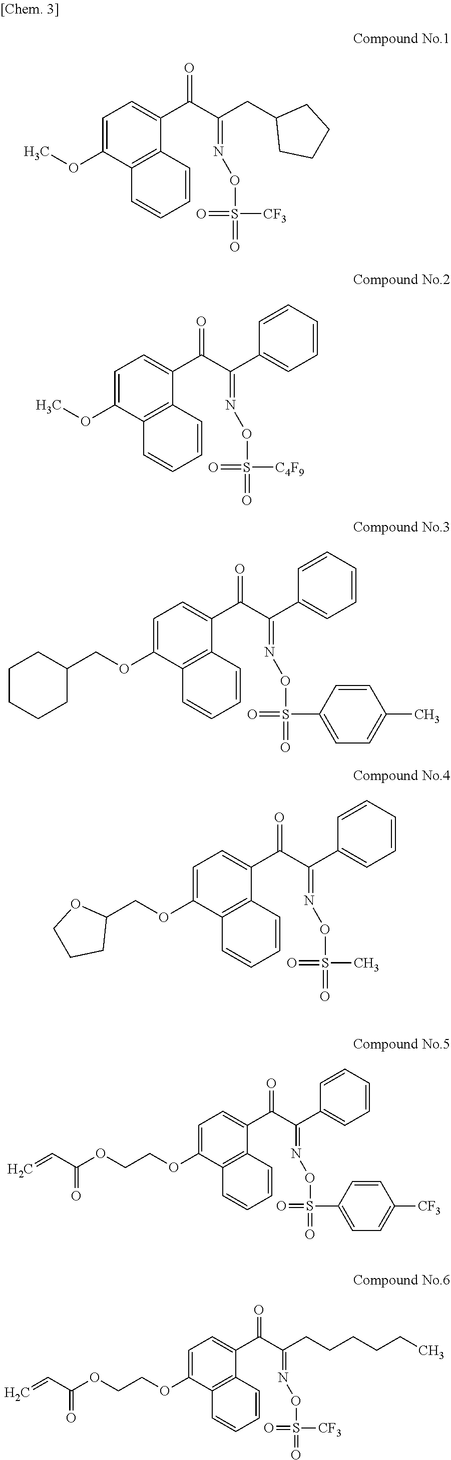

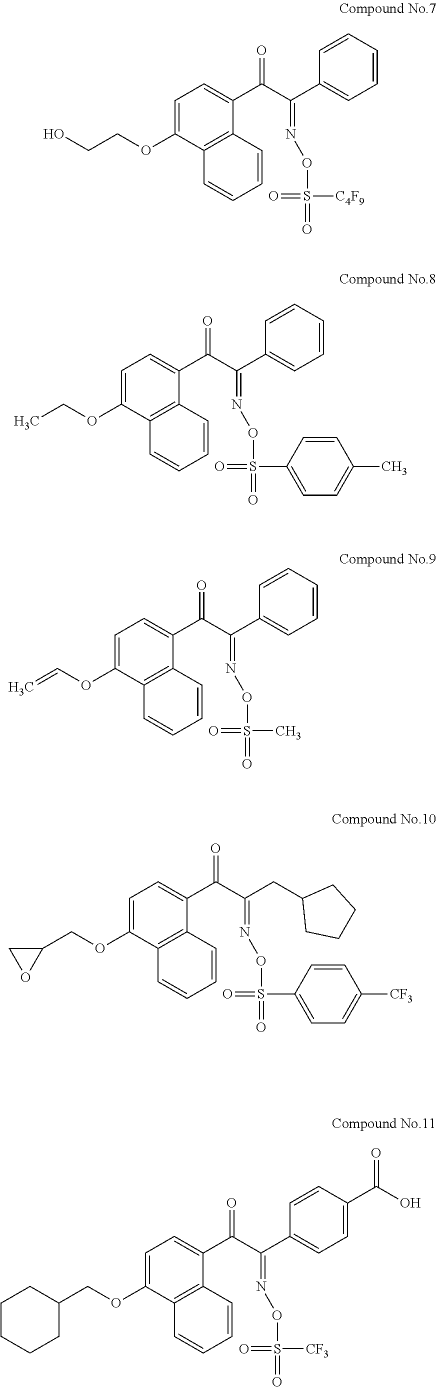

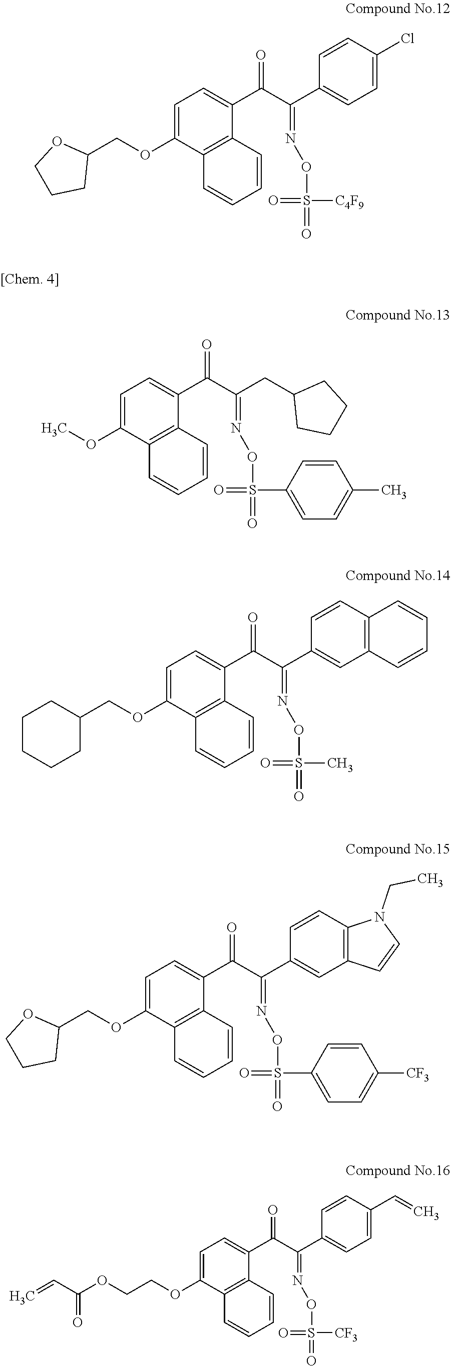

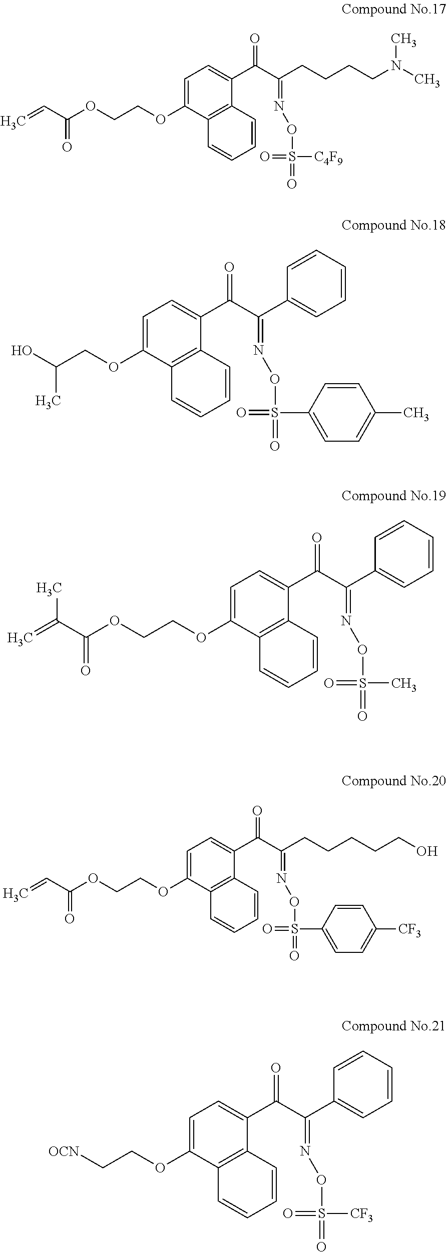

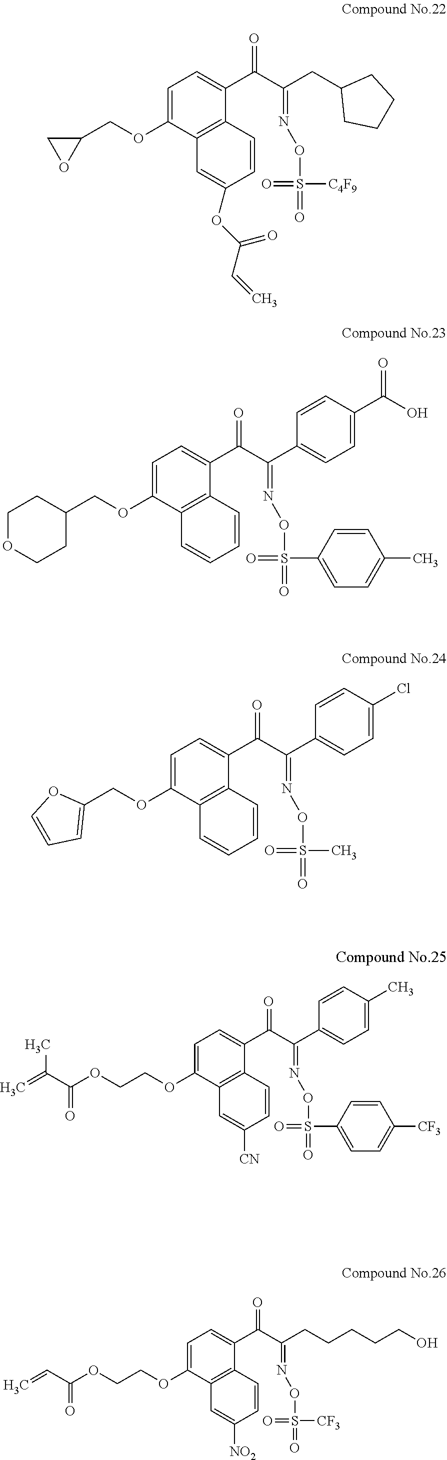

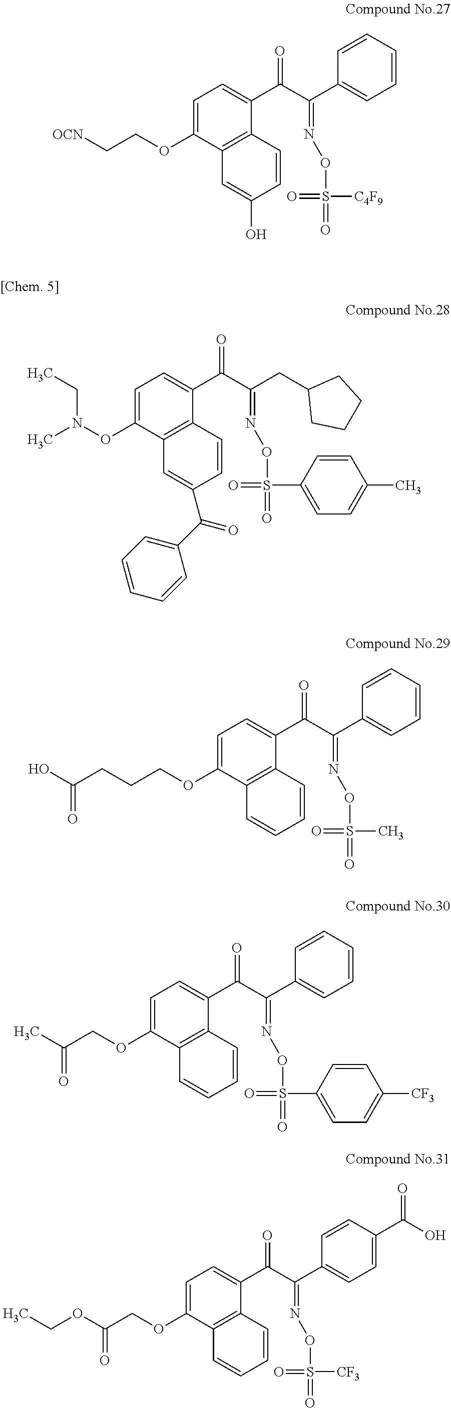

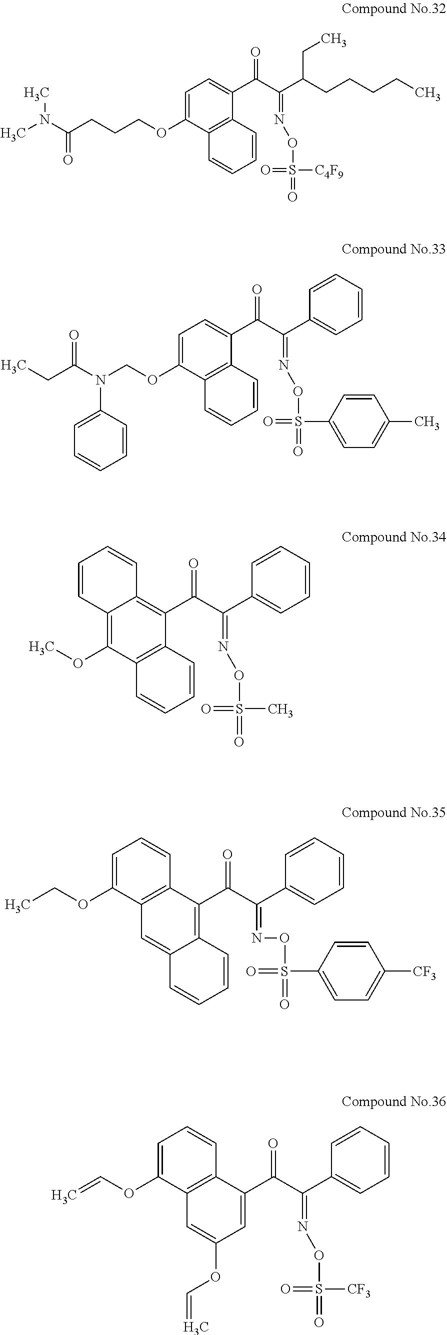

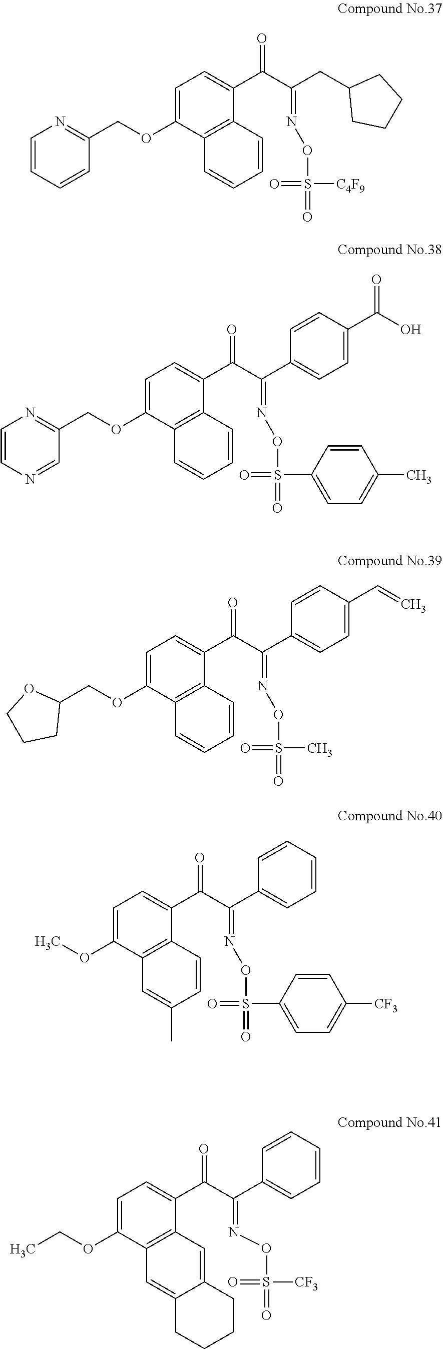

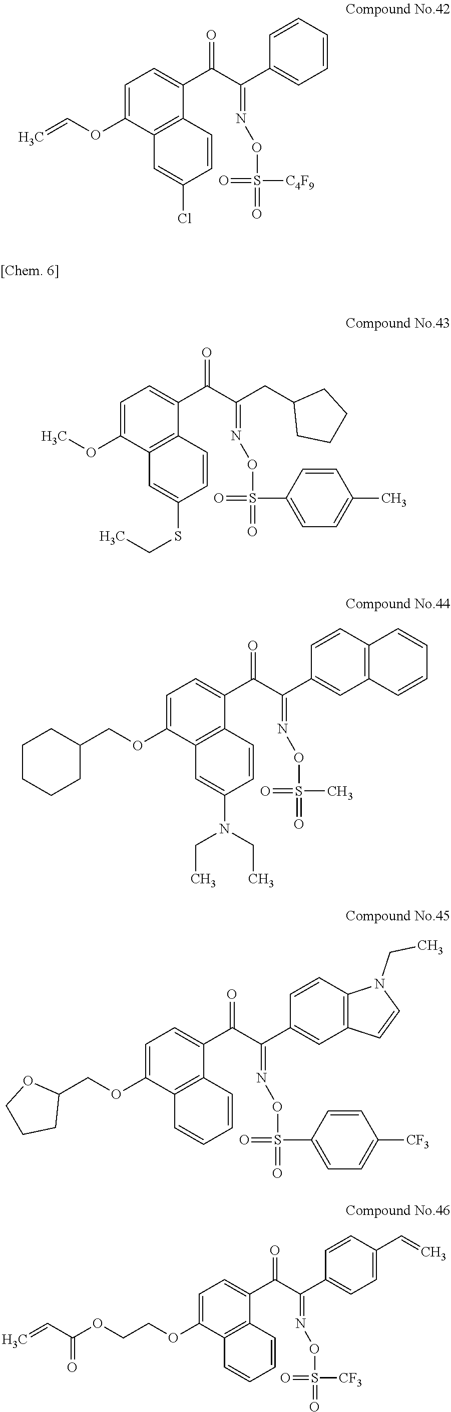

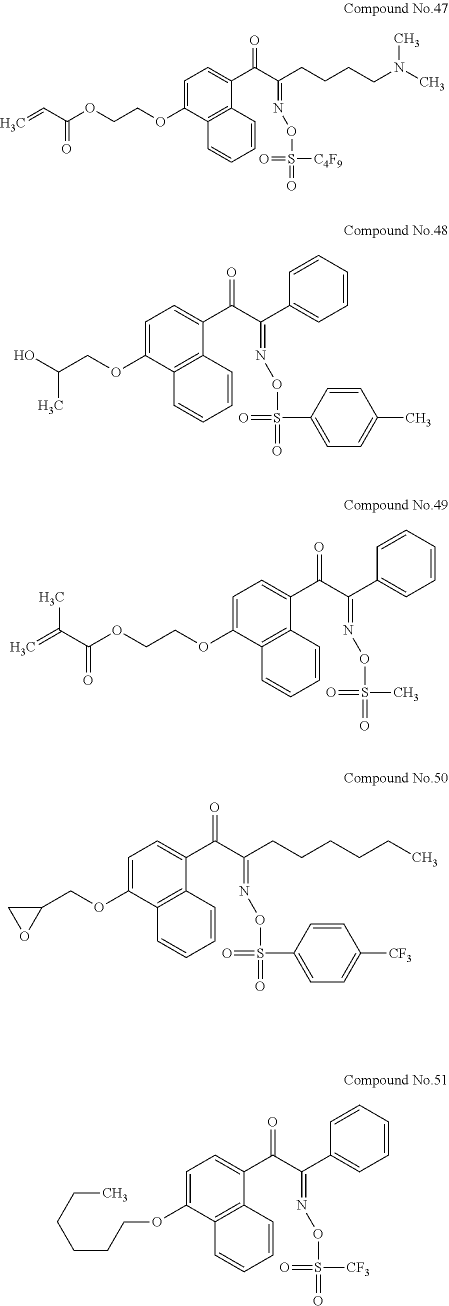

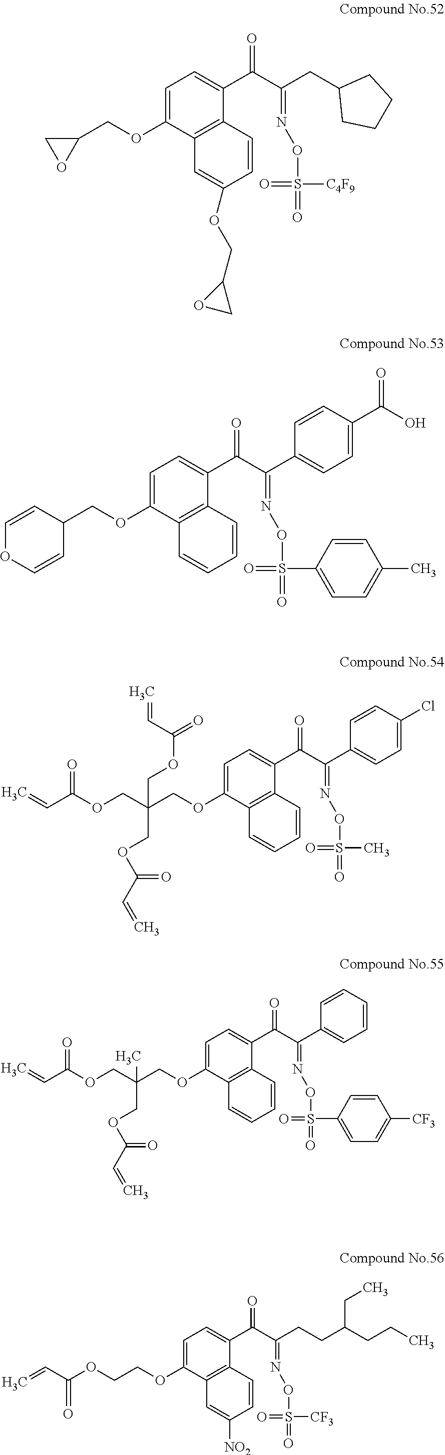

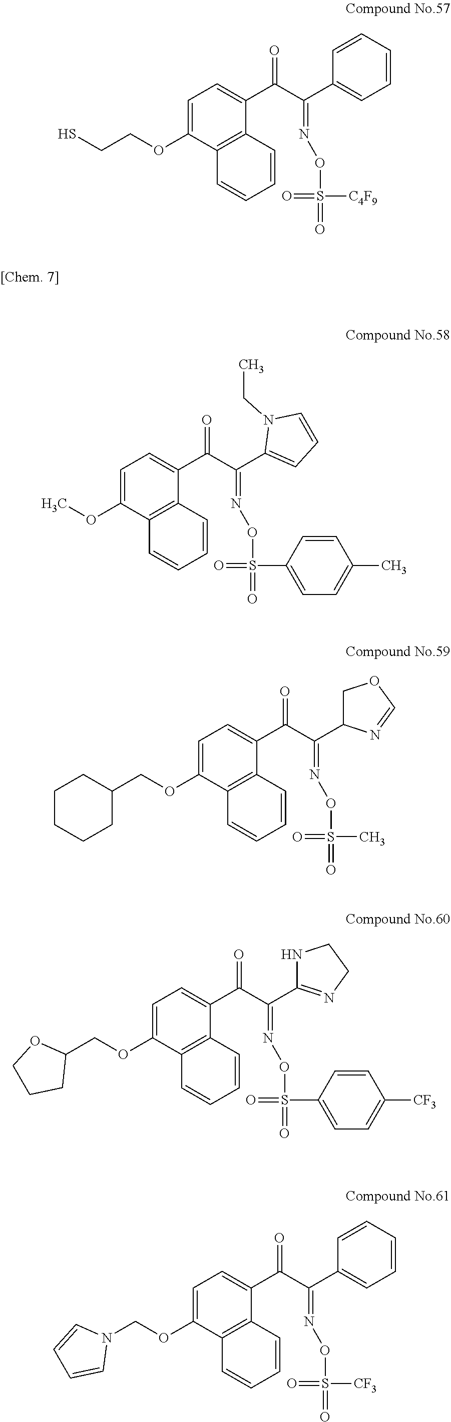

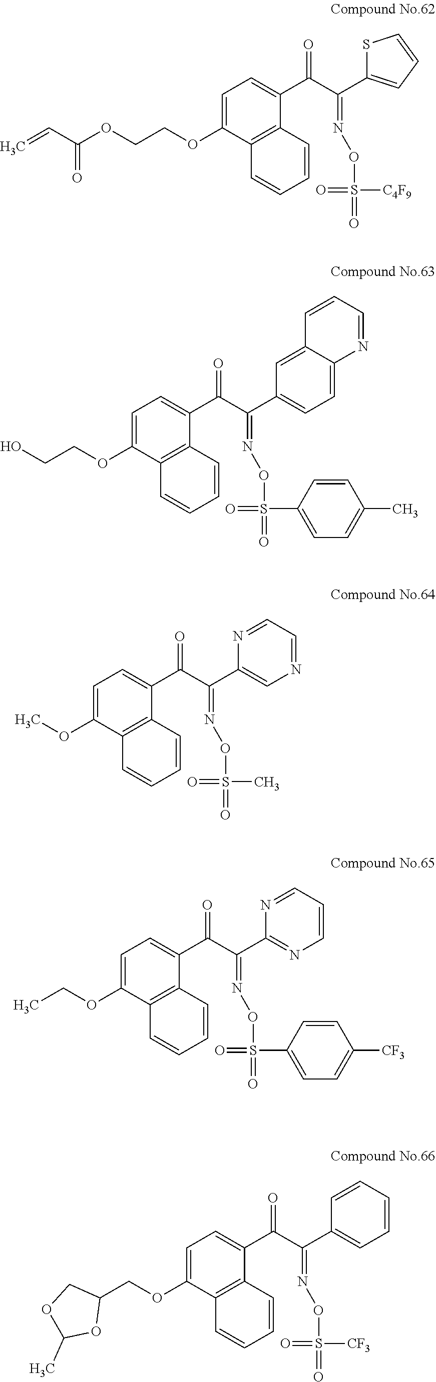

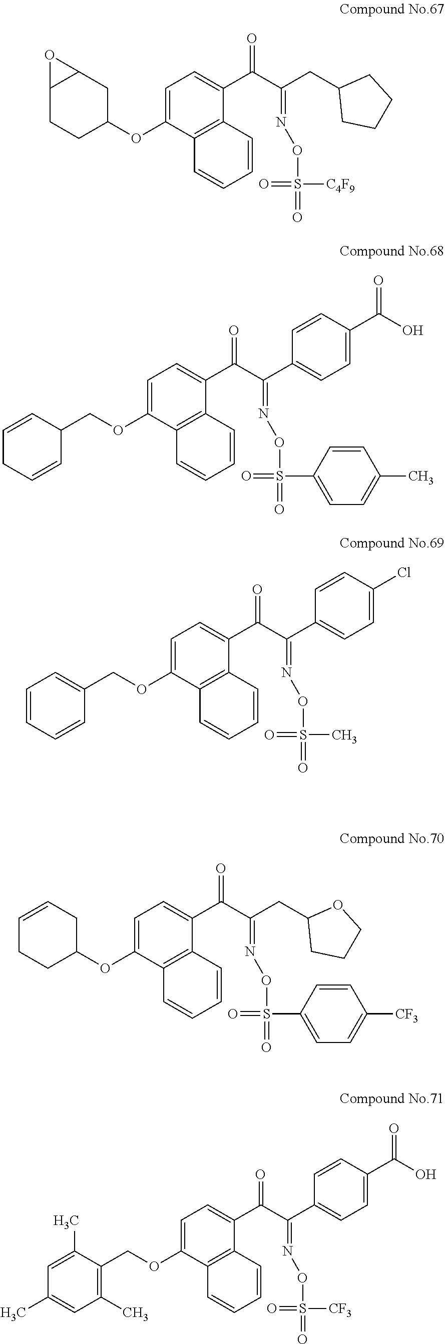

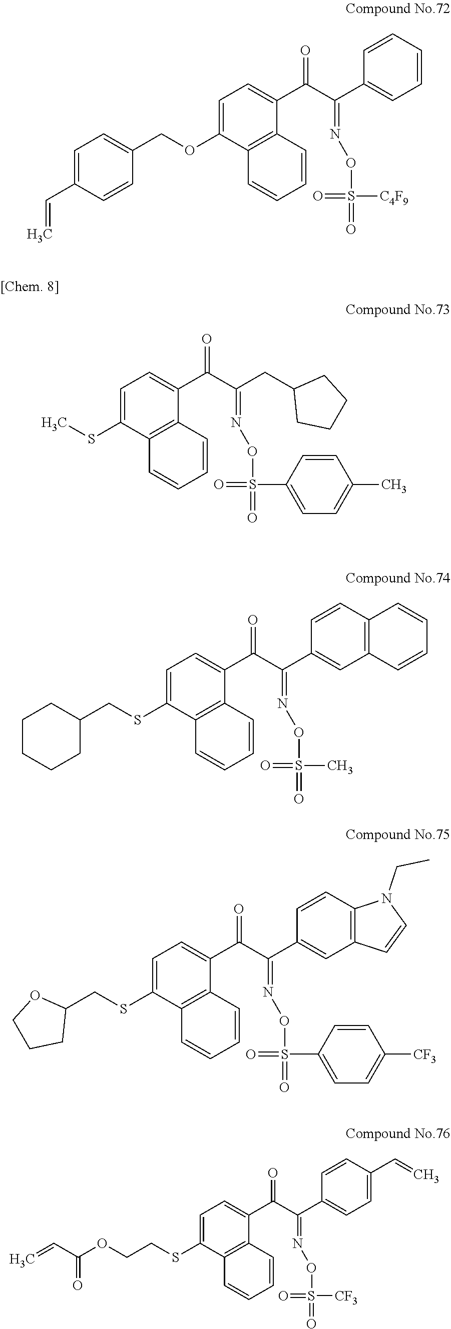

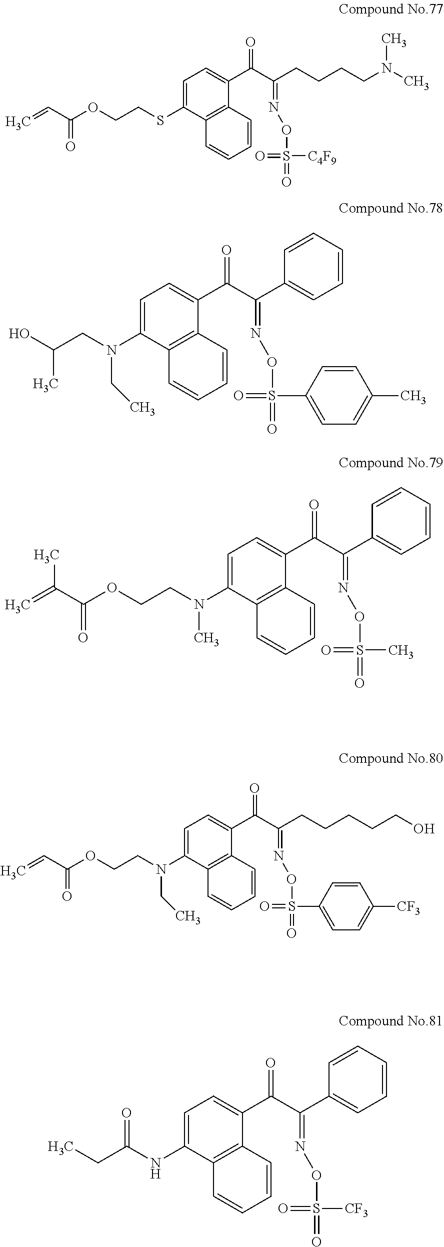

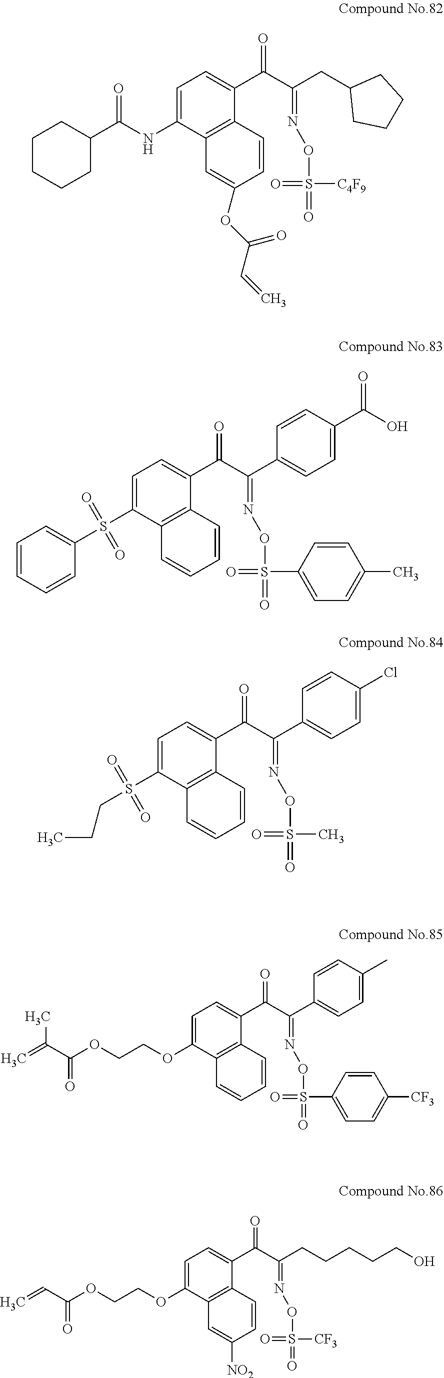

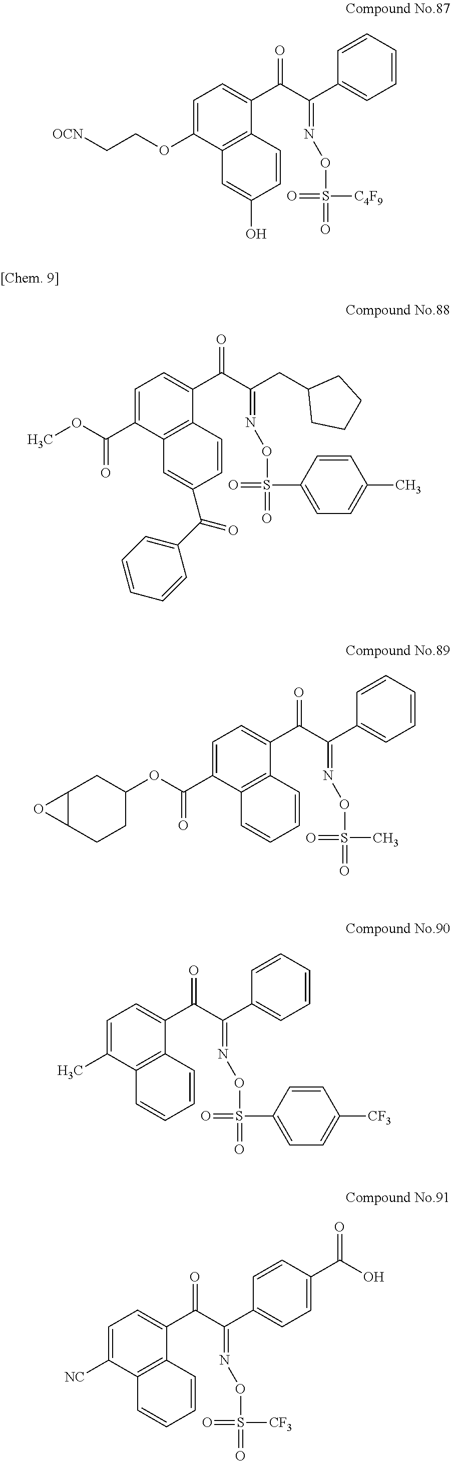

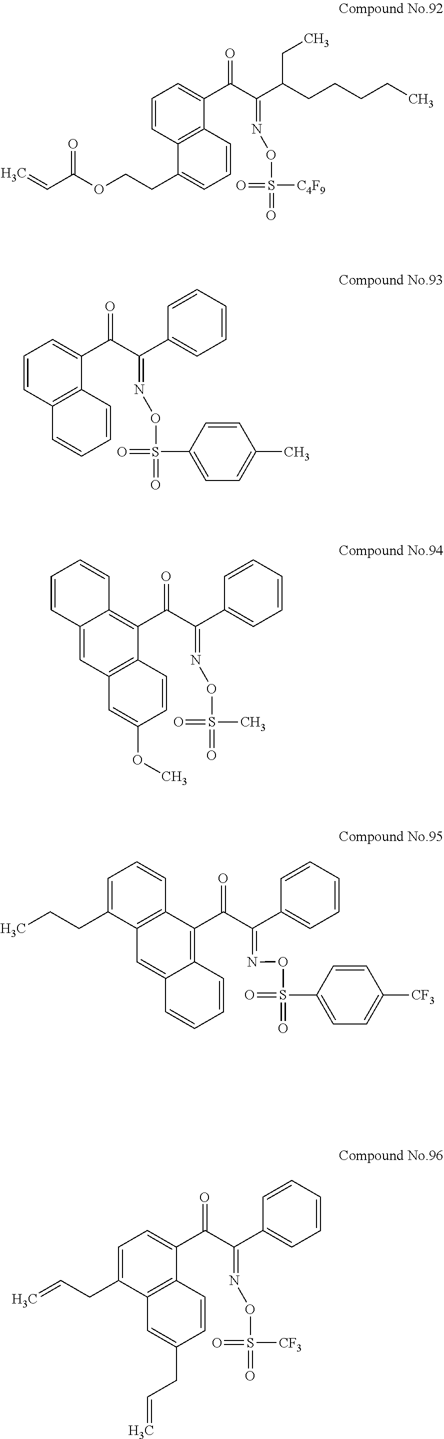

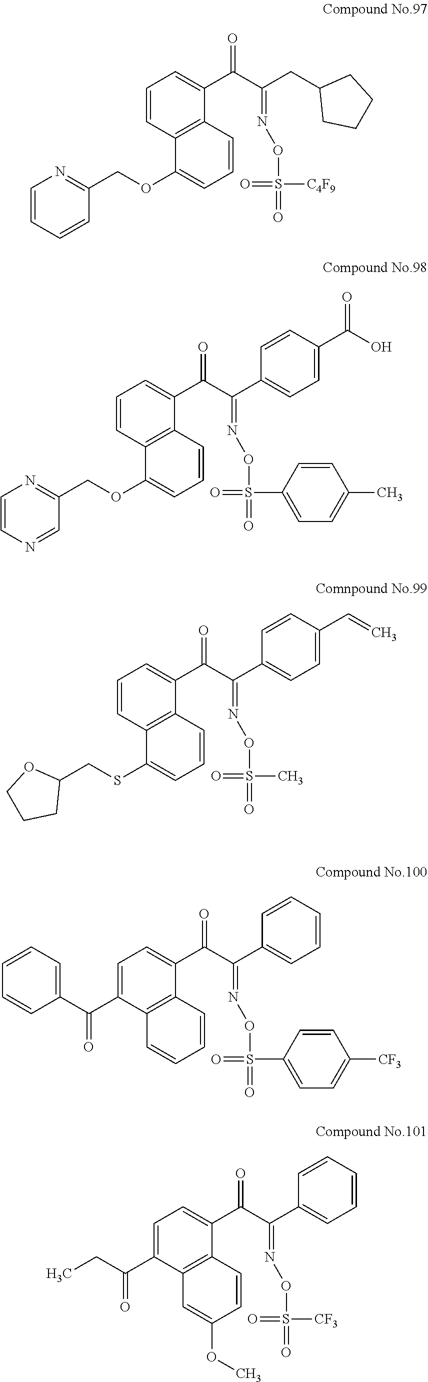

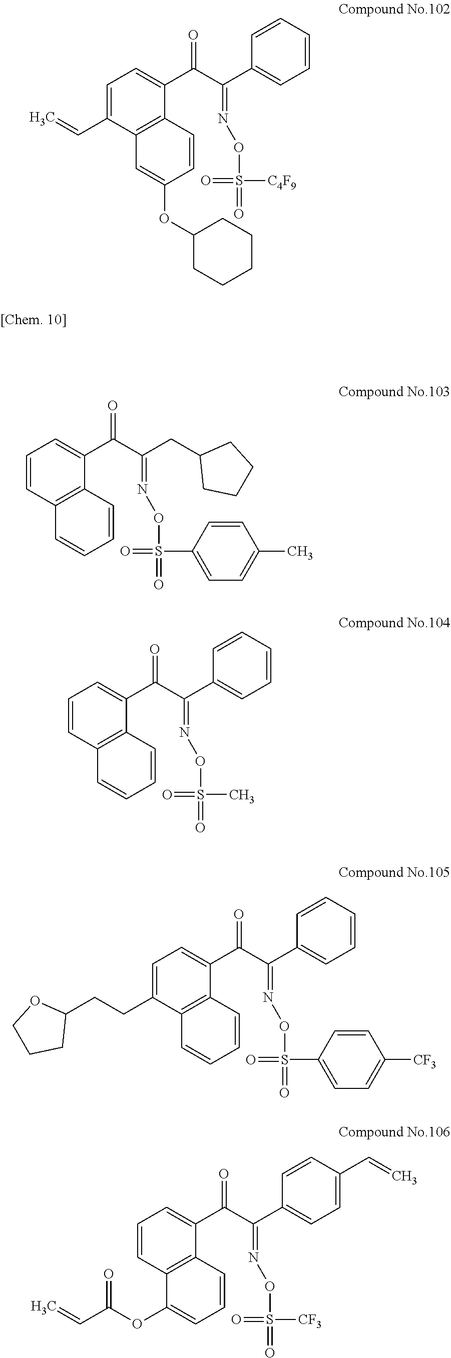

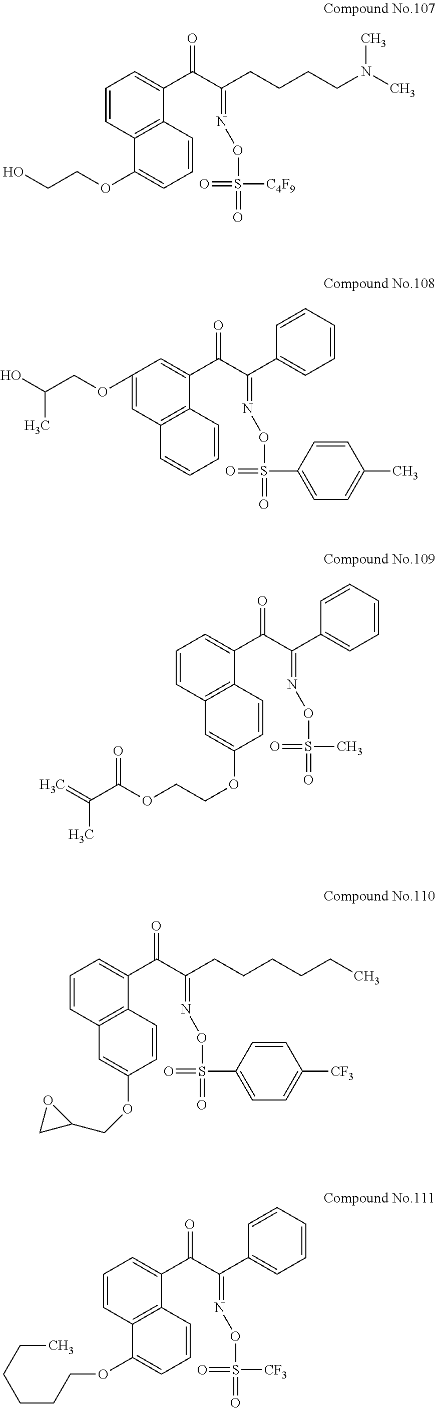

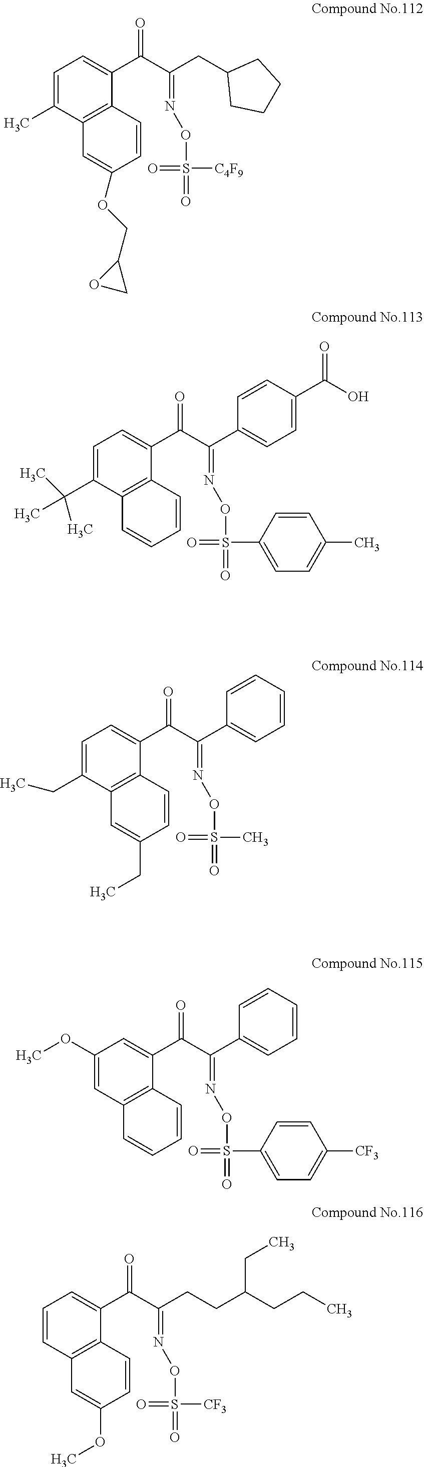

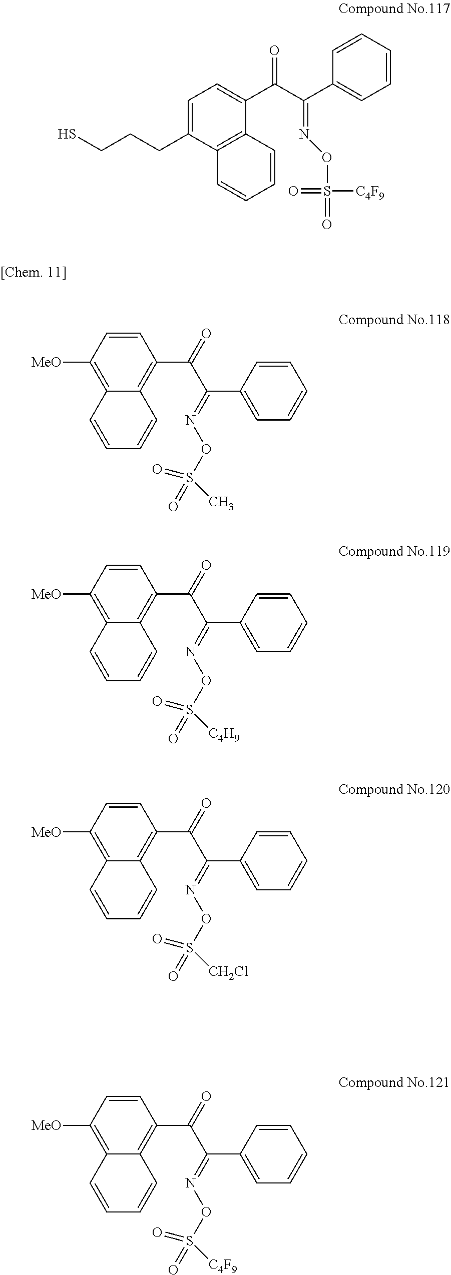

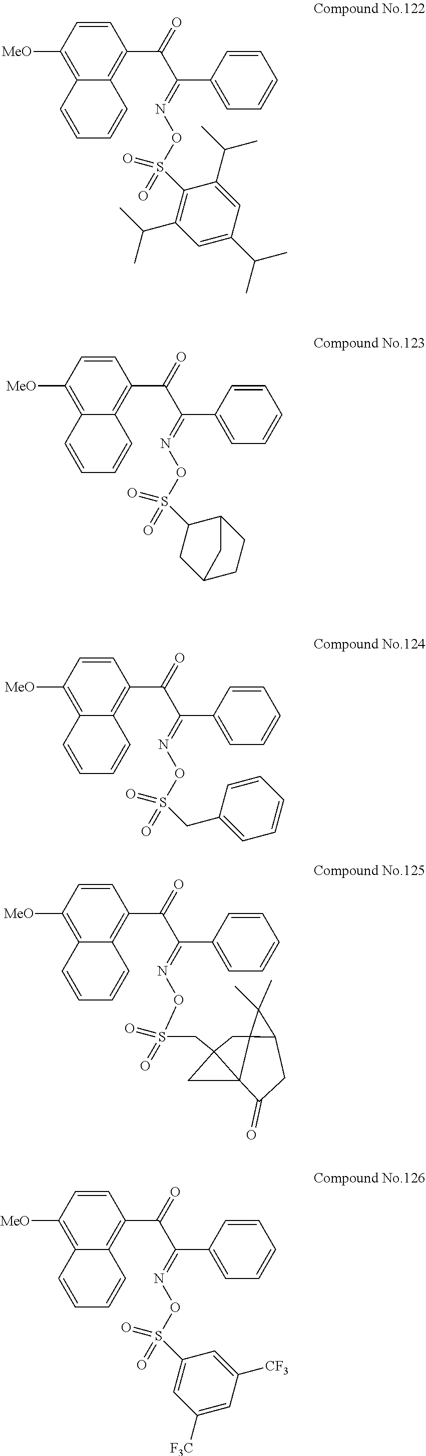 [SMILES: COc1ccc(C(=O)/C(=N/OS(=O)(=O)c2c(C(C)C)cc(C(C)C)cc2C(C)C)c2ccccc2)c2ccccc12 ; COc1ccc(C(=O)/C(=N/OS(=O)(=O)C2CC3CCC2C3)c2ccccc2)c2ccccc12 ; COc1ccc(C(=O)/C(=N/OS(=O)(=O)Cc2ccccc2)c2ccccc2)c2ccccc12 ; COc1ccc(C(=O)/C(=N/OS(=O)(=O)CC23CC24C(=O)CC2C(C)(C)C24C3)c2ccccc2)c2ccccc12 ; COc1ccc(C(=O)/C(=N/OS(=O)(=O)c2cc(C(F)(F)F)cc(C(F)(F)F)c2)c2ccccc2)c2ccccc12]

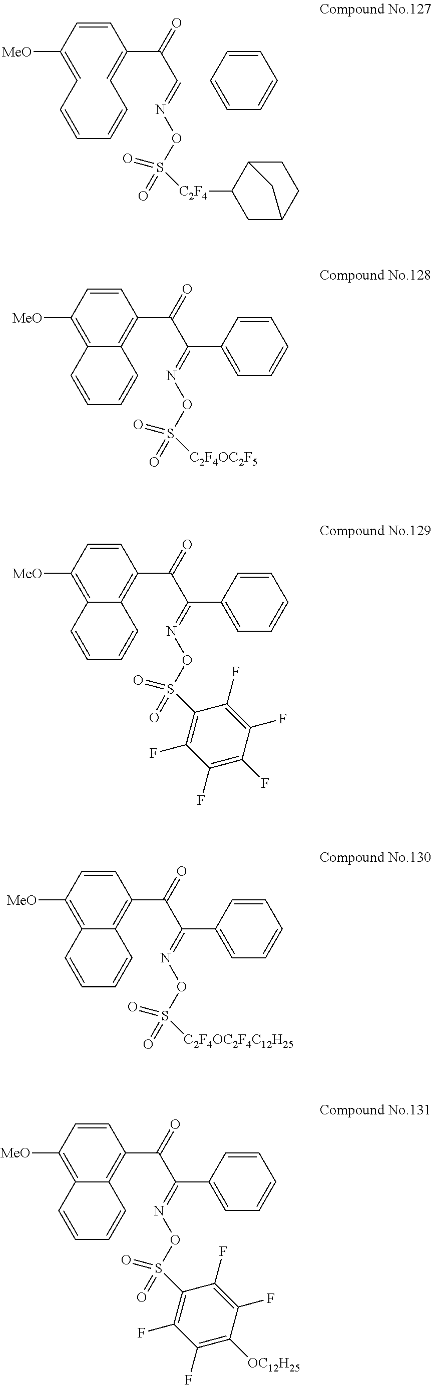

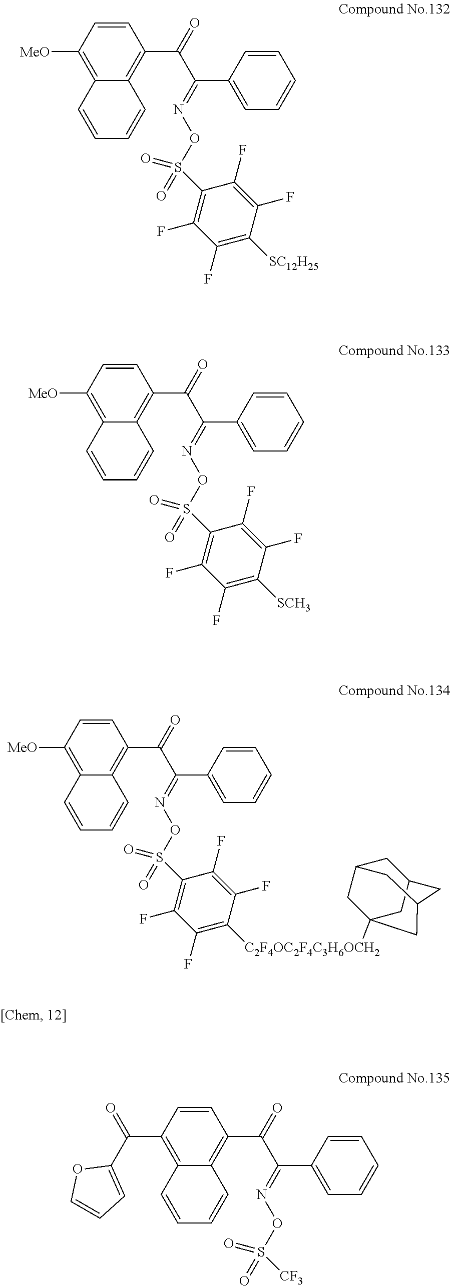

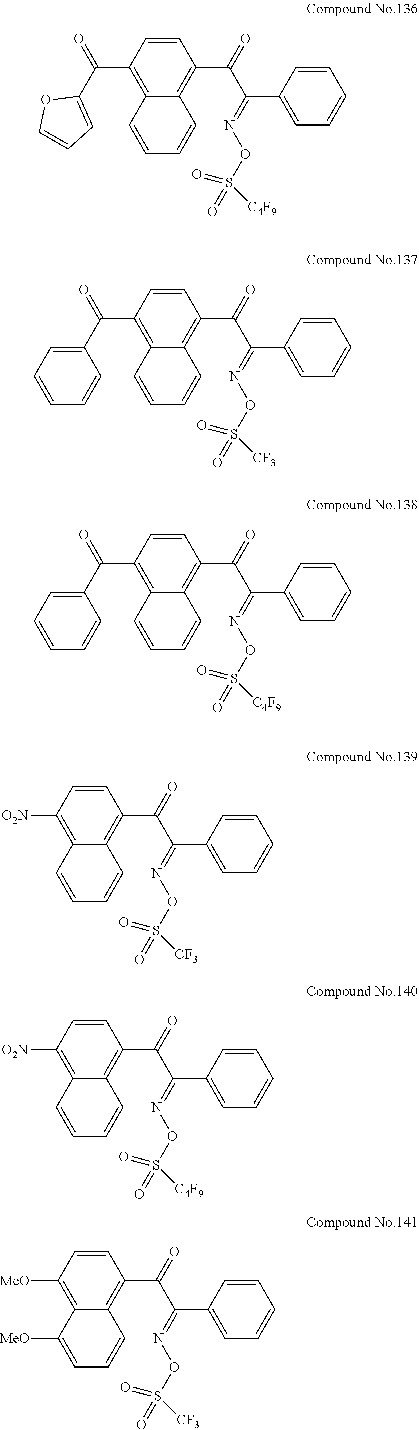

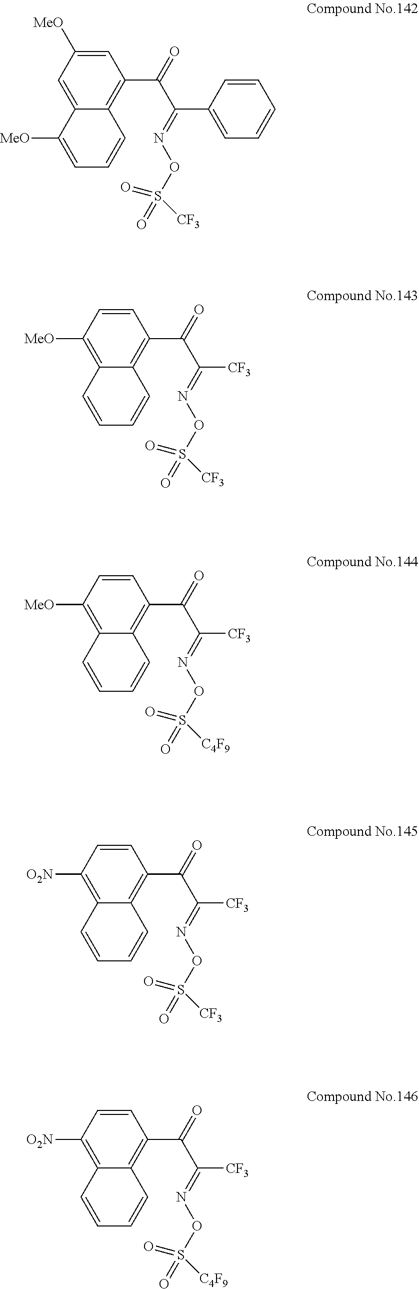

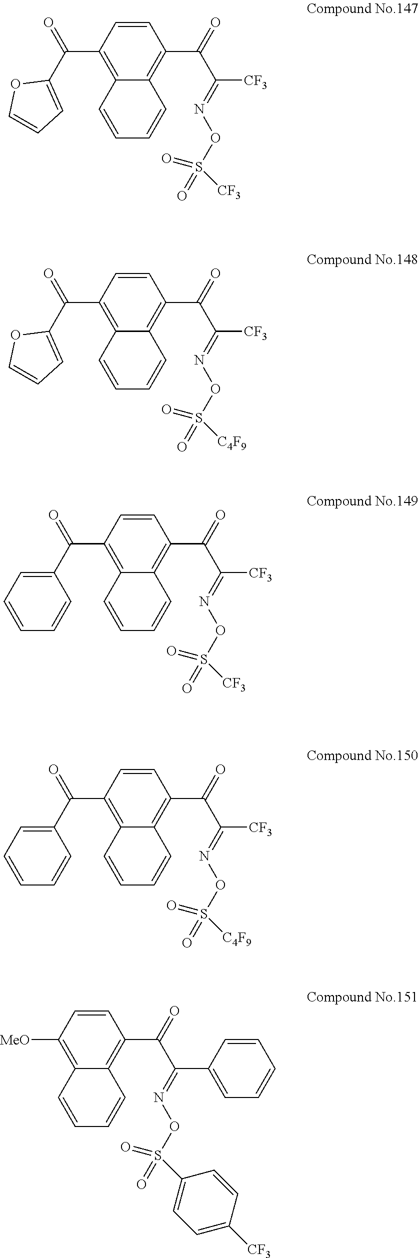

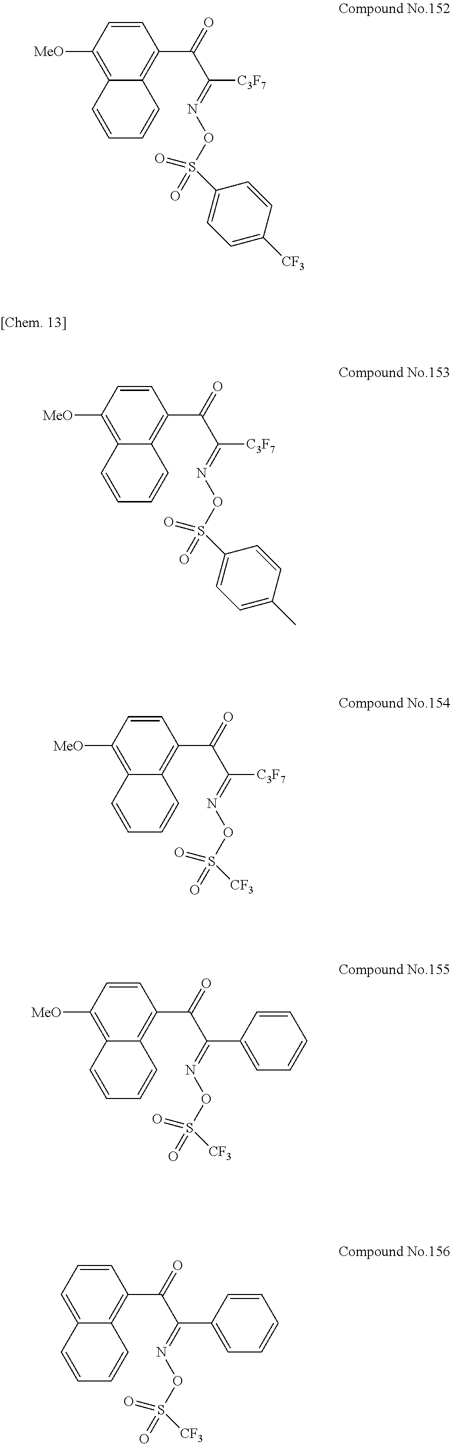

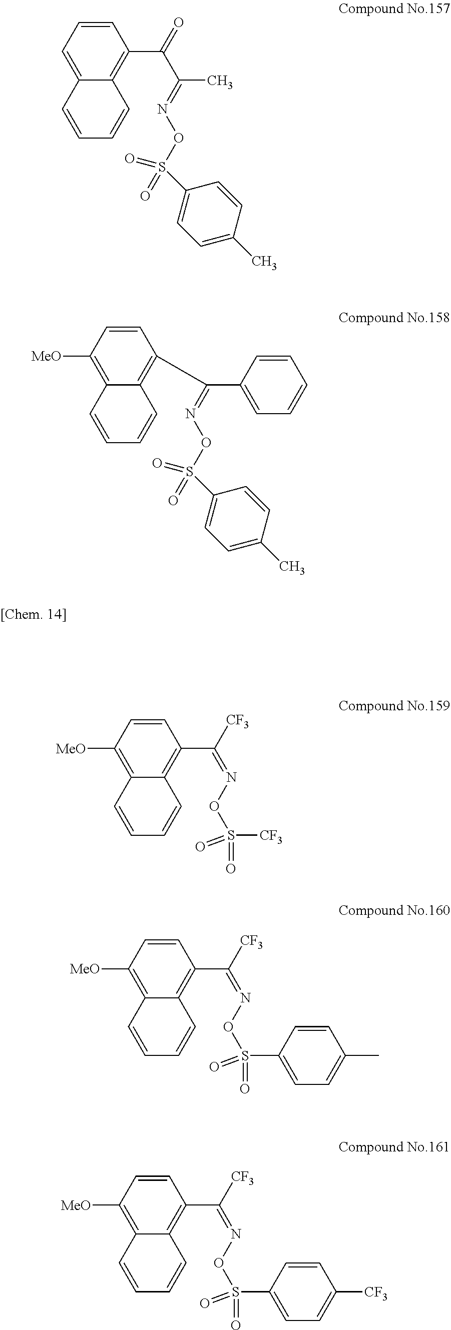 [SMILES: C/C(=N\OS(=O)(=O)c1ccc(C)cc1)C(=O)c1cccc2ccccc12 ; COc1ccc(/C(=N/OS(=O)(=O)c2ccc(C)cc2)c2ccccc2)c2ccccc12 ; COc1ccc(/C(=N\OS(=O)(=O)C(F)(F)F)C(F)(F)F)c2ccccc12 ; COc1ccc(/C(=N\OS(=O)(=O)c2ccc(C)cc2)C(F)(F)F)c2ccccc12 ; COc1ccc(/C(=N\OS(=O)(=O)c2ccc(C(F)(F)F)cc2)C(F)(F)F)c2ccccc12]

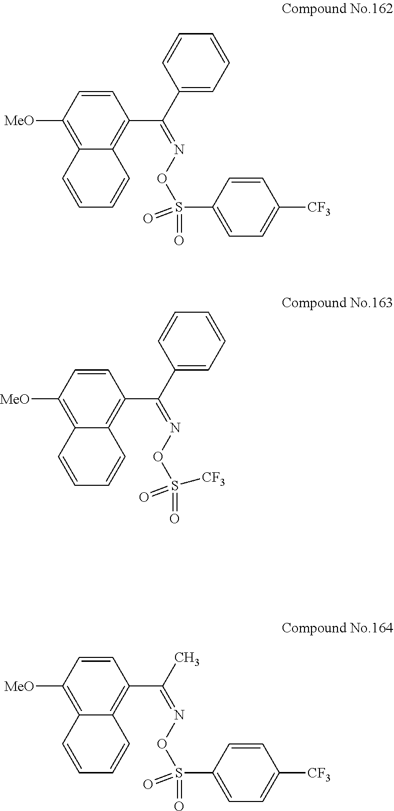 [SMILES: COc1ccc(/C(=N\OS(=O)(=O)c2ccc(C(F)(F)F)cc2)c2ccccc2)c2ccccc12 ; COc1ccc(/C(=N\OS(=O)(=O)C(F)(F)F)c2ccccc2)c2ccccc12 ; COc1ccc(/C(C)=N\OS(=O)(=O)c2ccc(C(F)(F)F)cc2)c2ccccc12]

The compound A can be prepared by any method using well-known chemical reactions selected to provide a compound structure as desired. For example, the method according to the reaction scheme below is proposed, in which a known, commercially available naphthalene compound and an acid chloride are caused to react with each other to form a ketone compound, the ketone compound is allowed to react with isobutyl nitrite to form an oxime compound, which is then allowed to react with a sulfonyl chloride compound to give a compound A. The reaction conditions for carrying out the method, such as the reaction temperature, reaction time, amounts of reactants, and the like are not particularly limited, and any known conditions can be used.

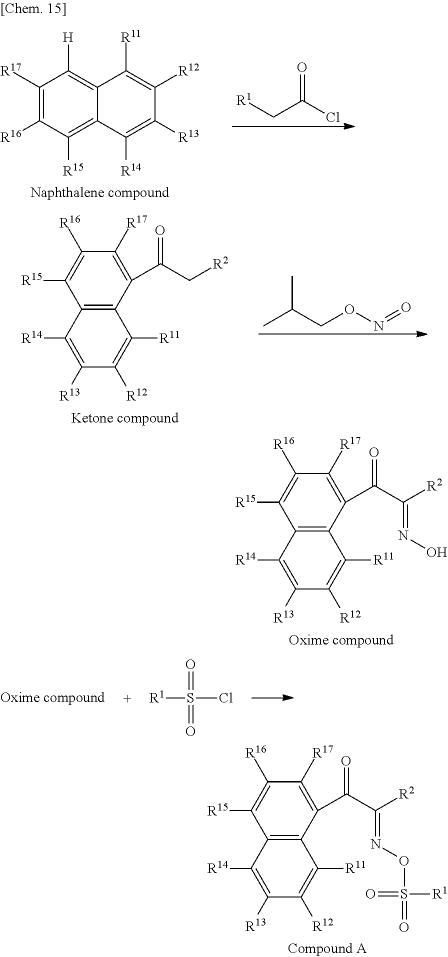

The compound A is capable of generating an acid.

Any process commonly applied to acid generators can be used to cause the compound A to generate an acid, including exposure to energy radiation or heat treatment, or a combination thereof conducted either successively or simultaneously.

Examples of the energy radiation include g-rays (436 nm), h-rays (405 nm), i-rays (365 nm), visible light, ultraviolet, far ultraviolet, X-rays, and charged particle radiation.

Sources of the radiation include low-, middle-, high- or ultrahigh-pressure mercury lamps, xenon lamps, metal halogen lamps, electron beam irradiators, X-ray irradiators, and lasers (e.g., argon lasers, dye lasers, nitrogen lasers, LEDs, and He—Cd lasers).

The heating temperature of the heat treatment preferably ranges from 700 to 450° C., more preferably from 150° to 300° C. The heating time of the heat treatment preferably ranges from 1 to 100 minutes. When the heat treatment is performed under these conditions, the effect of the compound A in providing a composition having reduced discoloration can be produced effectively.

Use applications of the compound A include acid generators. Specifically, the compound A is useful as a photoacid generator capable of generating an acid on exposure to energy radiation and a thermal acid generator capable of generating an acid on heating.

Applications of the acid generators include addition to a composition containing a resin component.

End use applications of the composition are not particularly limited and include optical filters, pigmented or non-pigmented coating materials, lining materials, adhesive, printing plates, insulating varnishes, insulating sheets, laminates, printed circuit boards; sealants for semiconductor devices, LED packages, liquid crystal filling holes, organic ELs, photonic devices, electrical insulation, electronic components, separation membranes, and the like; molding materials, putty, glass fiber impregnating agents, fillers; passivation layers for semiconductor devices, solar cells, and so on; interlayer dielectrics, surface protective films and printed circuit boards of TFTs, LCDs, Organic Els, printed circuit boards, and so on; color filters of color TV sets, PC monitors, PDAs, and CCD image sensors; electrodes of plasma display panels, printing inks, compositions for dental use, resins for stereolithography, both liquid and dry films, micromachine parts, glass fiber cable coatings, holographic recording materials, magnetic recording materials, optical switches, plating masks, etch masks, screen printing stencils, transparent conductive films for touch panels and the like, MEMS devices, nanoimprint materials, photofabrication such as 2D or 3D high density packaging for semiconductor packages, decorative sheets, artificial nails, glass-alternative optical films, electronic paper, optical discs, microlens arrays for use in projectors and lasers for optical communications, prism lens sheets for use in backlights of LCDs, Fresnel lens sheets for use in screens of projection TV sets and the like; the lens part of lens sheets, such as lenticular lens sheets, or backlights using these sheets; optical lenses of microlenses, taking lenses, and so on; optical devices, optical connectors, optical waveguides, insulating packing materials, heat shrinkable rubber tubes, O-rings, sealants for display devices, protective materials, optical fiber protective materials, pressure-sensitive adhesives, die bonding agents, highly heat-dissipating materials, highly heat-resistant sealing materials; parts of solar cells, fuel cells, and secondary batteries; solid electrolytes for batteries, insulating coating materials, photosensitive drums of copiers, gas separation membranes; civil engineering and construction materials, such as concrete protective materials, lining materials, soil injectable materials, sealing agents, cooling or heating storage media, glass coatings, and foam; medical materials, such as tubes, sealing materials, coating materials, sealants for sterilizers, contact lenses, oxygen enrichment membranes, and biochips; automotive parts, and parts of various machines.

In order to elicit a better effect on reduction of discoloration of the composition, the composition is preferably applied to parts requiring transparency. Such applications include optical filters, optical filter coatings, optical lenses, optical devices, optical connectors, optical waveguides, and transparent insulating layers of electronic circuits requiring transparency.

### B. Acid Generator

The acid generator according to the disclosure will then be described. The acid generator according to the disclosure is characterized by containing the compound A.

According to the disclosure, the acid generator, which contains the compound A, easily provides a composition and the like having reduced discoloration.

### B-1. Compound A

Any type of the compound A may be used in the acid generator of the present invention as long as it is capable of providing a composition and the like having reduced discoloration. The acid generator may contain one or more than one compound A.

The content of the compound A in the acid generator of the present invention can be decided as appropriate according to the type of the acid generator as long as the acid generator is capable of providing a composition and the like with reduced discoloration. For instance, the acid generator of the present invention may contain 100 parts by mass of the compound A per 100 parts by mass of its solids content, i.e., the solids content of the acid generator may be composed solely of the compound A. The content of the compound A in the acid generator may be less than 100 parts per 100 parts by mass of the solids content of the acid generator, i.e., the acid generator may contain the compound A and other optional components. For example, the content of the compound A in the acid generator may range from 20 to 99.99 parts by mass. As long as the content of the compound A is within that range, a composition and the like with reduced discoloration can be obtained easily.

In the case of the acid generator containing the optional component, the content of the compound A in the acid generator of the present invention is preferably at least 50 parts, more preferably 70 parts or higher, even more preferably 90 parts or higher, per 100 parts by mass of the solids content with the view of obtaining a composition having reduced discoloration more easily. As long as the upper limit of the content of the compound A is in that range, a composition and the like with reduced discoloration is obtained easily. With a view to facilitating particle size control of the acid generator, the content of the compound A in the acid generator of the present invention is preferably 99 parts by mass at the highest, more preferably 95 parts or lower, even more preferably 90 parts or lower, per 100 parts by mass of the solids content of the acid generator. With the content of the compound A falling within that range, a composition and the like with reduced discoloration can be obtained easily.

The term “solids content” as used herein refers to the total amount of all the components except a solvent. When the acid generator contains two or more compounds A, the term “content of the component A” refers to the total amount of the compounds A.

The description of the compound A given under the heading “A. Compound” applies equally to the compound A for use in the acid generator, so that the details of the compound A are omitted here.

### B-2. Other Optional Components

The optional components that may compose the acid generator together with the compound A include a solvent. The solvent is capable of dispersing or dissolving every component of the acid generator. Therefore, the compound A is not included in the category of “solvent” even if it is liquid at ordinary temperature (25° C.) and atmospheric pressure. The solvent to be used may be water or an organic solvent. Organic solvents are preferred for ease of dispersing or dissolving the compound A.

Examples of suitable solvents include carbonates, such as propylene carbonate and diethyl carbonate; ketones, such as acetone and 2-heptanone; polyhydric alcohols and derivatives thereof, such as ethylene glycol, propylene glycol, propylene glycol monoacetate, dipropylene glycol, and a monomethyl ether or monophenyl ether of dipropylene glycol monoacetate; cyclic ethers, such as dioxane; esters, such as ethyl formate and 3-methyl-3-methoxybutyl acetate; aromatic hydrocarbons, such as toluene and xylene; and lactones, such as γ-caprolactone and δ-caprolactone.

The amount of the solvent may range from 1 to 99 parts by mass per 100 parts by mass of the acid generator.

Optional components other than the solvent include the components hereinafter described under the subheadings “C-2. Resin component” and “C-3. Other optional components” under the heading “C. Composition”.

The content of the optional component(s) may be selected as appropriate to the end use and the like of the acid generator. For instance, the optional component content may be 50 parts or lower, preferably 10 parts or lower, per 100 parts by mass of the acid generator. With the content of the other optional components being within that range, it is easier to make the acid generator have an increased content of the compound A and to provide a composition and the like with reduced discoloration.

### B-3. Miscellaneous

The manner of preparing the acid generator is not restricted as long as the resulting acid generator has a desired content of the compound A. When the acid generator contains an optional component, any known mixing methods can be employed.

The use applications of the acid generator include addition to compositions containing a resin component. Specific end use applications of the acid generator are the same as those of the compound A described above under the heading “A. Compound”.

### C. Composition

The composition of the present disclosure will be described hereunder. The composition of the disclosure is characterized by containing the compound A and a resin component.

According to the disclosure, the composition containing the compound A shows reduced discoloration.

### C-1. Compound A

Any type of the compound A may be used in the composition of the present invention as long as the resulting composition has reduced discoloration. The composition may contain one or more than one compound A.

The content of the compound A in the composition of the present invention may be selected appropriately according to the type and so forth of the resin component to be combined with.

In view of the ease of producing the effect of the compound A in reducing discoloration of the composition, the content of the compound A in the composition of the present invention is, for instance, preferably 0.05 to 100 parts, more preferably 0.05 to 20 parts, by mass per 100 parts by mass of the resin component.
In terms of the ease of achieving the effect of the compound A in providing a composition having high sensitivity and reduced discoloration, the content of the compound A in the composition of the invention is, for instance, preferably 0.001 to 20 parts by mass per 100 parts by mass of the solids content of the composition.

In view of the ease of obtaining the effect of the compound A in making a composition having high sensitivity and reduced discoloration, the content of the compound A in the composition of the invention is, for instance, preferably 0.001 to 20 parts by mass per 100 parts by mass of the composition.

When the composition contains two or more compounds A, the term “content of the component A” refers to the total amount of the compounds A.

The description of the compound A given under the heading “A. Compound” applies equally to the compound A for use in the composition, so that the details of the compound A are omitted here.

### C-2. Resin Component

The resin component may be either a polymer or a component capable of forming a polymer. The resin component is a component other than the compound A.

The resin component may be either reactive or non-reactive with the acid generated from the compound A but is preferably acid-reactive because the effect of the compound A in generating an acid and providing a composition having reduced discoloration can be obtained effectively.

The acid-reactive resin component is preferably selected from an acid-curable component capable of curing through polymerization or crosslinking induced by the action of the acid generated by the compound A and an acid-decomposable component that increases its solubility in a developer by the action of the acid generated by the compound A.

The composition containing an acid-curable resin component provides a cured product having reduced discoloration. The composition containing an acid-decomposable resin component shows reduced discoloration in non-developed portions, i.e., portions where solubility in a developer has not changed. Specifically, in some of the use applications recited above, the composition undergoes a heat treatment step, a light irradiation step, and the like in addition to the acid generation step. In such cases, an acid is generated from part of the compound A, and the compound A having generated the acid exhibits an inhibitory effect on oxidative deterioration that is thought to be a cause of discoloration. As a result, the non-developed portion is protected from discoloration. Furthermore, the acid-decomposable component is prevented from oxidative deterioration that is though to be a cause of discoloration, whereby the acid-decomposable component easily changes in developer solubility.

The acid-curable component is exemplified by cationically polymerizable compounds, including cyclic ethers, such as epoxy compounds and oxetane compounds, vinyl ethers, vinyl compounds, styrenes, spiro ortho esters, bicyclo ortho esters, spiro orthocarbonates, lactones, oxazolines, aziridines, cyclosiloxanes, ketals, cyclic acid anhydrides, lactams, and aryldialdehydes. Polymerizable or crosslinkable polymers and oligomers having the above recited polymerizable group in their side chain are also included. These acid-curable components may be used either individually or in combination of two or more thereof.

Specific examples of the cationically polymerizable compounds include the acid reactive organic substances described in WO 2017/130896 and the compounds described as cationically polymerizable compounds in WO 2014/084269 and WO 2016/132413.

A mixture of a crosslinkable resin and a crosslinking agent also serves as an acid-curable component.

Examples of the crosslinkable resin include polyhydroxystyrene and its derivatives; polyacrylic acid and its derivatives; polymethacrylic acid and its derivatives; copolymers of at least two of hydroxystyrene, acrylic acid, methacrylic acid, and their derivatives; copolymers of at least two of hydroxystyrene, styrene, and their derivatives; copolymers of at least three of cycloolefins and their derivatives, maleic anhydride, and acrylic acid and its derivatives; copolymers of at least three of cycloolefins and their derivatives, maleimide, and acrylic acid and its derivatives; polynorbornene; at least one high-molecular polymer selected from the group consisting of ring-opening metathesis polymers; and the high-molecular polymers partially substituted by an acid-labile group having capability of controlling alkali solubility. The polymers having a repeating unit derived from hydroxystyrene, such as polyhydroxystyrene, are exemplified by the phenolic hydroxy-containing resins (QN) described in JP 2018-112670A.

Examples of the crosslinkable resins include the resist base resins described in WO 2017/130896, the resins of which the solubility in an alkaline developer changes by the action of an acid component (A) described in JP 2003-192665A, and the resins described as alkali-soluble resins in JP 2004-323704A, Claim 3 and JP 10-10733A.

Any crosslinking agent may be used as long as it is capable of crosslinking the molecules of the crosslinkable resin in the presence of an acid. Such crosslinking agents include compounds reactive with an acidic group, such as a phenolic hydroxy or a carboxy, of the crosslinkable resin in the presence of an acid, such as epoxy-, hydroxy-, alkoxy-, methylol-, or carboxymethyl-containing compounds. Specifically, the crosslinking agents described in JP 2016-169173A and JP 2018-112670A are useful.

The acid-decomposable component is not limited as long as it increases in solubility in a developer by the action of an acid generated from the compound A, including resins having an acidic group, such as phenolic hydroxy, carboxy, or sulfonyl, with a part or all of the hydrogens of the acidic groups protected with a protective group. Examples of the resin having an acidic group include the above described crosslinkable resins that are used as an acid-curable component together with a crosslinking agent. The positively-working, chemically amplified resins described in JP 2018-112670A are also useful. The protective group may be any protective group capable of protecting the acidic group, including the protective groups of JP 2016-169173A, the acid-labile groups of WO 2017/130896, and the acid-dissociable groups of JP 2018-112670A. The developer as referred to above will be described under the heading “F. Method for forming patterned coating”.

In addition to the acid-curable and acid-decomposable components, also useful as the acid-reactive component is a component that undergoes any reaction by the action of an acid, such as a resin having an alkali-soluble group that is insolubilized by an acid. Examples of such a resin include an acid-insolubilizable resin that undergoes, for example, an intra- or intermolecular crosslinking reaction through acid-catalyzed dehydration condensation between a hydroxyl and a carboxyl or, as shown in the reaction scheme below, between carboxyls. Examples of the acid-insolubilizable resin that undergoes acid-catalyzed dehydration condensation between carboxyls include a resin having a phthalic acid structure of which the carboxyls undergo dehydration condensation in the presence of an acid as shown below.

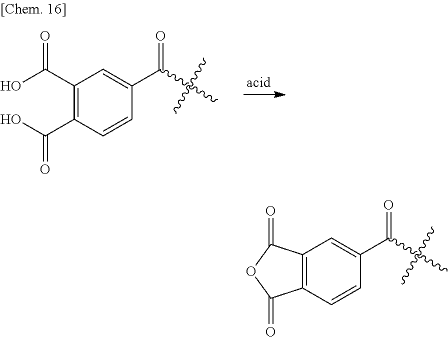

The resin component that is non-reactive with an acid may be a component that does not react with the acid generated from the compound A, specifically a component that does not undergo a change, such as cure, decomposition, or change in solubility in an alkaline developer, by the action of the acid generated from the compound A. Examples of the non-acid-reactive component include thermoplastic resins, such as polyolefin resins, polybutadiene resins, polystyrene resins, polystyrene-butadiene resins, and polystyrene-olefin resins.

The content of the resin component in the composition of the present invention is not critical as long as the effect in reducing discoloration is produced and is selected as appropriate to the type of the resin component.

The content of the resin component in the composition of the present invention may be, for instance, 10 parts by mass or higher per 100 parts by mass of the solids content of the composition. With the view to effective achievement of the effect on reduction of discoloration, the content is preferably 30 to 99.9 parts by mass, more preferably 50 to 99.9 parts by mass. The content of the resin component in the composition of the present invention may be, for instance, 10 parts by mass or higher per 100 parts by mass of the composition. With the view to effective achievement of the effect on reduction of discoloration, the content is preferably 30 to 99.9 parts by mass, more preferably 50 to 99.9 parts by mass.

### C-3. Solvent

The composition may further contain a solvent. The solvent is capable of dispersing or dissolving each component of the composition. Therefore, neither the compound A nor the resin component is included in the category of “solvent” even if it is liquid at ordinary temperature (25° C.) and atmospheric pressure. The solvent to be used may be water or an organic solvent. Organic solvents are preferred for ease of dispersing or dissolving the compound A. Examples of useful organic solvents may be the same as those enumerated above under the heading “B. Acid generator”. The content of the solvent in the composition of the present invention can be selected as appropriate according to the intended use of the composition. For example, the solvent content may be 1 to 99 parts by mass per 100 parts by mass of the composition.

### C-4. Other Optional Components

If desired, the composition may contain other optional components. The other optional components may be chosen according to the intended use and the like of the composition. Examples of useful optional components include benzotriazole, triazine, or benzoate UV absorbers; phenol, phosphorus, or sulfur antioxidants; antistatics, such as cationic, anionic, nonionic, or amphoteric surfactants; flame retardants, such as halogen compounds, phosphoric ester compounds, phosphoric amide compounds, melamine compounds, fluororesins or metal oxides, melamine (poly)phosphates, and piperazine (poly)phosphates; lubricants, such as hydrocarbons, fatty acids, aliphatic alcohols, aliphatic esters, aliphatic amides, and metal soaps; colorants, such as dyes, pigments, and carbon black; inorganic siliceous additives, such as fumed silica, fine silica particles, silica stone, diatomaceous earths, clay, kaolin, diatomaceous earth, silica gel, calcium silicate, sericite, kaolinite, flint, feldspar powder, vermiculite, attapulgite, talc, mica, minnesotite, pyrophyllite, and silica; fillers, such as glass fiber and calcium carbonate; crystallizing agents, such as nucleating agents and crystallization accelerators; silane coupling agents; rubber elasticity imparting agents, such as flexible polymers; and sensitizers. Acid diffusion controlling agents are also used as an optional component, including amine compounds, amido-containing compounds, urea compounds, and nitrogen-containing heterocyclic compounds.

Examples of the sensitizers include the compounds described as spectral sensitizers in JP 2008-506749A. Examples of the acid diffusion controlling agents include the compounds described in JP 2019-8300A under the heading “[D] Acid diffusion controller”.

The total content of these optional components in the composition of the present invention may be up to 50 parts by mass per 100 parts by mass of the composition.

### C-5. Miscellaneous

Any known technique can be used to prepare the composition as long as the aforementioned components are mixed in a desired ratio. For example, the compound A is dissolved or dispersed in a solvent, and a resin component is added thereto to make the composition.

### D. Cured Product

The cured product according to the disclosure is a cured product of the above-described composition wherein the resin component is an acid-curable component.

According to the disclosure, a cured product having reduced discoloration is obtained by using the above composition.

The cured product of the present invention is characterized by being obtained from the composition wherein the resin component is an acid-curable component. The cured product results from the cure of the acid-curable component and contains a polymer resulting from polymerization of the acid-curable component or a crosslinking product resulting from crosslinking of the acid-curable component. The details of the composition containing the acid-curable component are omitted here, since the relevant description under the heading “C. Composition” equally applies thereto.

The cured product may have any plan-view shape selected as appropriate to the intended end use and the like, such as a dot or line pattern.

The cured product has the same use applications as enumerated under the heading “A. Compound”.

The method for forming the cured product is not particularly limited as long as the composition is cured into a desired shape. For instance, the method described below under the heading “E. Method for producing cured product” may be followed.

### E. Method for Producing Cured Product

The cured product production method of the present disclosure is characterized by including the step of curing the above-described composition wherein the resin component is an acid-curable component.

According to the disclosure, a cured product with reduced discoloration can easily be obtained by using the composition.

### E-1. Curing Step

The curing step of the disclosure is a step of curing the composition. Any process capable of curing the acid-curable component can be adopted to cure the composition, including a process causing the compound A to generate an acid.

Any process that causes the compound A to generate a desired amount of an acid is usable, including exposure to energy radiation, heat treatment, and a combination thereof conducted either successively or simultaneously. With respect to these processes, refer to the description given under the heading “A. Compound”.

The details of the composition containing an acid-curable component as the resin component are the same as those described under the heading “C. Composition” and are therefore omitted herein.

### E-2. Other Steps

Where necessary, the cured product production method of the disclosure may further include other steps in addition to the curing step. Examples of the other steps include a development step in which an unpolymerized portion of a coating formed of the composition is removed after the curing step to form a patterned cured product, a post-baking step in which the cured product is heat-treated, a prebaking step in which the composition is heated to remove the solvent prior to the curing step, and a coating step in which the composition is applied to form a coating film to be cured.

The development step may be carried out by applying a developer, such as an alkaline developer, to the unpolymerized portion to remove the unpolymerized portion. Any commonly used alkaline developer may be used, including a tetrametylammonium hydroxide (TMAH) aqueous solution, a potassium hydroxide aqueous solution, or a potassium carbonate aqueous solution. A commonly used solvent developer may also be used, such as propylene glycol monomethyl ether acetate (PEGMEA) and cyclohexanone.

Development using a developer can be performed in any known manner as long as a portion to be developed is brought into contact with the developer, such as showering, spraying, or dipping. The development may be effected at any time after the curing step.

The post-baking step can be carried out under any heating conditions selected to improve the strength and the like of the cured product after the curing step. For example, the heating may be conducted at 2000 to 250° C. for 20 to 90 minutes.

The pre-baking step can be carried out under any heating conditions selected to remove the solvent from the composition, for example, at 700 to 150° C. for 30 to 300 seconds.

The coating step may be carried out by applying the composition to a substrate by any known methods, such as spin coating, roller coating, bar coating, die coating, curtain coating, printing, and dipping. The substrate may be selected as appropriate to the intended use of the cured product, including soda glass, quartz glass, semiconductor substrates, wiring boards, metals, paper, and plastics.

The cured product once formed on a substrate may be used as released from the substrate or transferred to another substrate.

### F. Process for Forming Patterned Coating

The process of patterned coating formation according to the disclosure includes an acid generation step in which the above-described composition wherein the resin component is an acid-decomposable component is cured into a coating and causing the compound A in the coating to generate an acid and a patterned coating formation step in which a part of the coating after the acid generation step is developed to form a patterned coating.

According to the disclosure, use of the above-described composition enables formation of a patterned coating excellent in dimensional accuracy and the like.

### F-1. Acid Generation Step

The acid generation step is to cause the compound A present in the coating formed of the composition to generate an acid. This step may be achieved by any technique to produce a desired amount of an acid from the compound A, such as exposure to energy radiation, heat treatment, and a combination thereof conducted either successively or simultaneously. With respect to these processes, refer to the description given under the heading “A. Compound”.

The portion of the coating where an acid is to be generated is preferably a plan-view portion of the coating. In that case, the subsequent step of patterned coating formation is easier to perform.

The plan-view shape and thickness of the coating are selected appropriately according to the intended use and the like of the patterned coating.

The composition contains an acid-decomposable component as the resin component. The details of the composition of this type are the same as those described under the heading “C. Composition” and are therefore omitted herein.

### F-2. Patterned Coating Formation Step

This step is to develop a part of the coating after the acid generation step to form a patterned coating.

The development can be accomplished by using a developer. For the details of the developer and developing method, refer to the relevant description under the heading “E. Method for producing cured product”.

### F-3. Other Steps

If necessary, the patterned coating formation method of the disclosure may further include other steps in addition to the acid generation step and the patterned coating formation step. Examples of the other steps include a coating step in which a coating film of the composition is formed prior to the acid generation step and a prebaking step in which the coating film is heated to remove the solvent prior after the coating step. For the details of the coating formation step and the prebaking step, refer to the relevant description under the heading “E. Method for producing cured product”.

### F-4. Miscellaneous

The details of the patterned coating formed by the above-described method and the use applications thereof are the same as those described under the heading “A. Composition”.

The disclosure is not limited to the foregoing embodiments. It should be understood that the above embodiments are only for illustration and that any technical idea having substantially the same constitution as the concept described in the claims of the disclosure and achieving the same effects and advantages is included in the scope of the disclosure.

### EXAMPLES

The disclosure will now be illustrated in greater detail by way of Examples, but it should be noted that the disclosure is not deemed to be limited thereto.

### Example 1

In a 200 mL four-necked flask were placed 10.0 g (63.2 mmol) of 1-methoxynaphthalene, 110 g of EDC, and 9.5 g (69.5 mmol) of zinc chloride in a nitrogen atmosphere, followed by stirring. To the mixture was dropwise added 11.7 g (75.9 mmol) of phenylacetyl chloride, followed by stirring at room temperature for 2 hours. After completion of the reaction, ethyl acetate and ion-exchanged water were added to the reaction mixture for oil-water separation. The separated oily phase was worked up by washing with water, dehydration, filtration, and solvent removal in that order and further purified by column chromatography on silica gel to give intermediate 1-A.

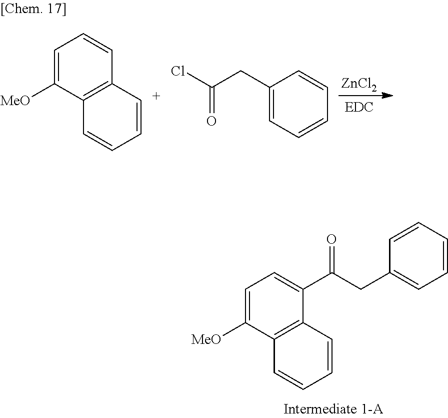

### Step 2:

In a 200 mL four-necked flask were placed 10.7 g (38.7 mmol) of intermediate 1-A and 25.0 g of DMF in a nitrogen atmosphere, followed by stirring to dissolve, followed by cooling. To the solution were added dropwise 4.4 g (42.6 mmol) of 35% hydrochloric acid and 6.0 g (58.1 mmol) of isobutyl nitrite, followed by stirring for 2 hours. After completion of the reaction, ethyl acetate and ion-exchanged water were added to the reaction mixture for oil-water separation. The resulting oily phase was washed with water, crystallized with toluene, filtered, and dried to give intermediate 1-B.

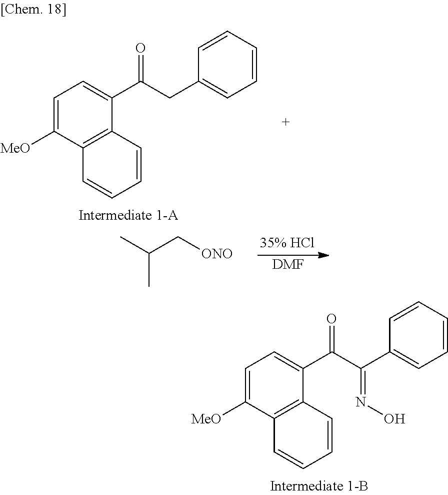

### Step 3:

In a 100 mL four-necked flask were put 6.9 g (22.6 mmol) of intermediate 1-B, 21 g of dichloromethane, and 5.2 g (27.1 mmol) of p-toluenesulfonyl chloride in a nitrogen atmosphere, followed by stirring, followed by cooling. Then, 2.5 g (24.9 mmol) of triethylamine was added thereto dropwise at or below 10° C., followed by stirring for 2 hours. After completion of the reaction, ion-exchanged water was added to the reaction mixture for oil-water separation. The separated oily phase was washed with water, crystallized from methanol, filtered, and dried to yield compound A1 represented by formula (A1) below as a solid. The resulting product was identified to be compound A1 by ¹H-NMR and IR analyses. The ¹H-NMR and IR results are shown in Tables 1 and 2.

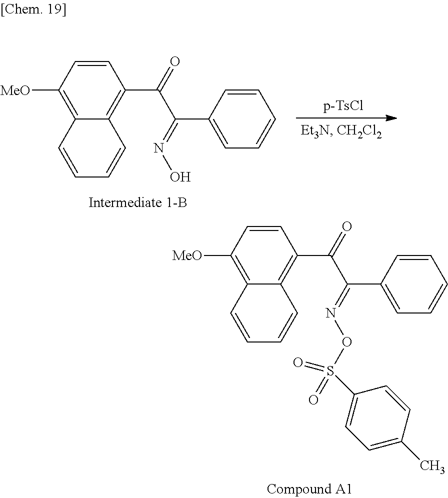 [SMILES: COc1ccc(C(=O)/C(=N/O)c2ccccc2)c2ccccc12 ; COc1ccc(C(=O)/C(=N/OS(=O)(=O)c2ccc(C)cc2)c2ccccc2)c2ccccc12]

### Example 2—Synthesis of Compound A2

Compound A2 represented by formula (A2) below was synthesized in the same manner as for compound A1, except for replacing p-toluenesulfonyl chloride with trifluoromethanesulfonic anhydride. Identification of the resulting solid product was confirmed by ¹H-NMR and IR analyses.

### Example 3—Synthesis of Compound A3

Compound A3 represented by formula (A3) below was synthesized in the same manner as for compound A2, except for replacing trifluoromethanesulfonic acid with nonafluoromethanesulfonic acid. Identification of the resulting solid product was confirmed by ¹H-NMR and IR analyses.

### Example 4—Synthesis of Compound A4

Compound A4 represented by formula (A4) below was synthesized in the same manner as for compound A2, except for replacing 1-methoxynaphthalene with naphthalene. Identification of the resulting solid product was confirmed by ¹H-NMR and IR analyses.

### Example 5—Synthesis of Compound A5

Compound A5 represented by formula (A5) below was synthesized in the same manner as for compound A1, except for replacing 1-methoxynaphthalene with naphthalene and replacing phenylacetyl chloride with propionyl chloride. Identification of the resulting solid product was confirmed by ¹H-NMR and IR analyses.

### Example 6—Synthesis of Compound A6

### Step 1:

Intermediate 6-A was synthesized in the same manner as for intermediate 1-A, except for replacing phenylacetyl chloride with benzoyl chloride.

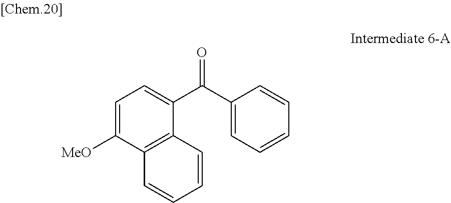 [SMILES: COc1ccc(C(=O)c2ccccc2)c2ccccc12]

### Step 2:

In a 200 mL four-necked flask were placed 5.4 g (20.6 mmol) of intermediate 6-A, 17.1 g of pyridine, and 2.1 g of hydroxylamine hydrochloride in a nitrogen atmosphere, followed by stirring. Acetic acid and ion-exchanged water were added to the reaction mixture for oil-water separation. The separated oily phase was worked up by washing with water, dehydration, filtration, and solvent removal to give intermediate 6-B.

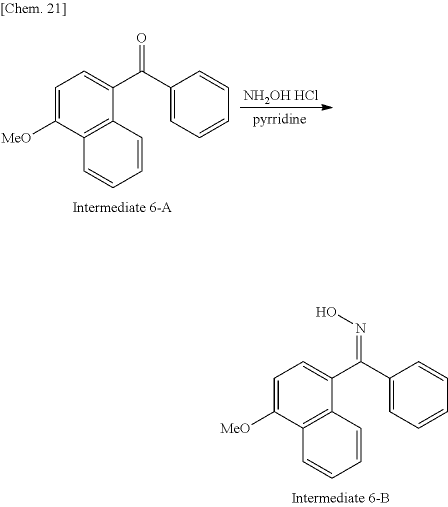

### Step 3:

Compound A6 represented by formula (A6) below was synthesized in the same manner as in step 3 for the synthesis of compound A1, except for using intermediate 6-B in place of intermediate 1-B. Identification of the resulting solid product was confirmed by ¹H-NMR and IR analyses.

TABLE 1
¹H-NMR (solvent: CDCl₃)

| | Chemical Shift (ppm) (multiplicity, number of protons) |
|---|---|
| Compound A1 | 9.47 (d, 1H), 8.37 (d, 1H), 7.86 (m, 3H), 7.76 (t, 1H), |
| | 7.61 (m, 3H), 7.44 (t, 1H), 7.35 (m, 4H), 6.75 (d, 1H), |
| | 4.08 (s, 3H), 2.46 (s, 3H) |

TABLE 2

| | IR Absorption Spectrum (cm⁻¹) |
|---|---|
| Compound A1 | 2254, 1659, 1573, 1514, 1468, 1446, 1427, |
| | 1379, 1284, 1254, 1228, 1193, 1179, 1096, |
| | 913, 846, 814, 802, 772, 743 |

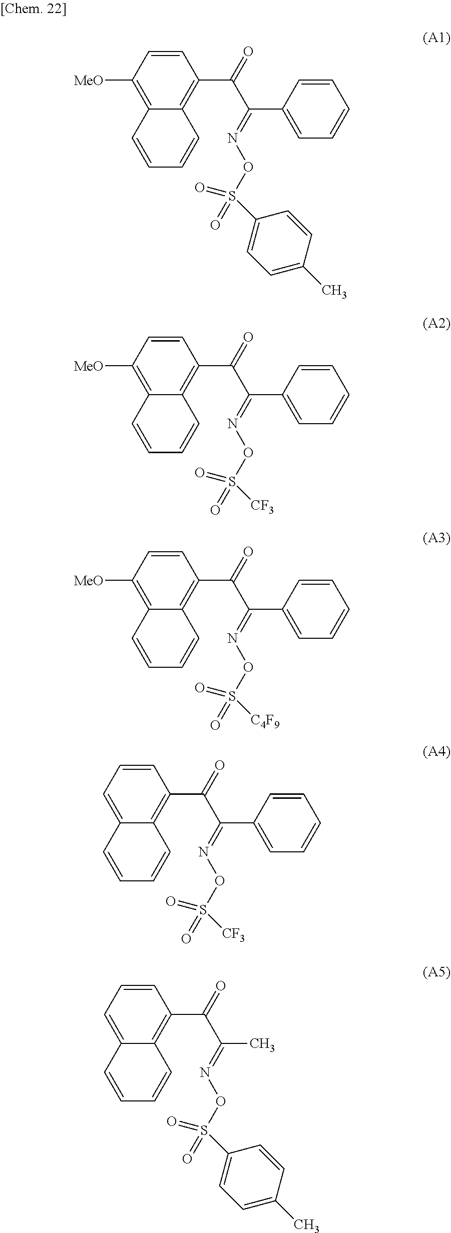

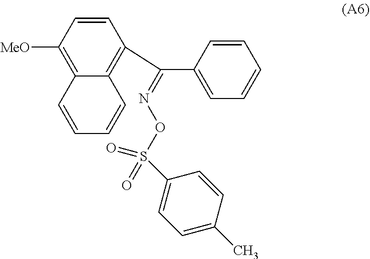 [SMILES: COc1ccc(/C(=N/OS(=O)(=O)c2ccc(C)cc2)c2ccccc2)c2ccccc12]

### Examples 7 to 12 and Comparative Examples 1 to 4

An epoxy compound, a novolak resin, an acid generator, and a surfactant were added to propylene glycol monomethyl ether acetate (PEGMEA) in accordance with the compounding ratio shown in Table 3, in which the amounts are given in part by mass. The mixture was stirred at 25° C. for 1 hour to prepare a composition (a PEGMEA solution having a solids content of 25 mass %). The components used are as follows.

Epoxy compound: EPPN-201 from Nippon Kayaku Co., Ltd.; a crosslinking agent as an acid-curable component.
Novolak resin: BRG-558 from Showa Denko Co., Ltd.; a crosslinkable resin as an acid-curable component.
Surfactant: FZ2122 from Toray Dow Corning Co., Ltd.
Compounds A1 to A6: compounds A1 to A6 prepared in Examples 1 to 6.
Compounds B1 to B3: compounds represented by formulae (B1) to (B3):

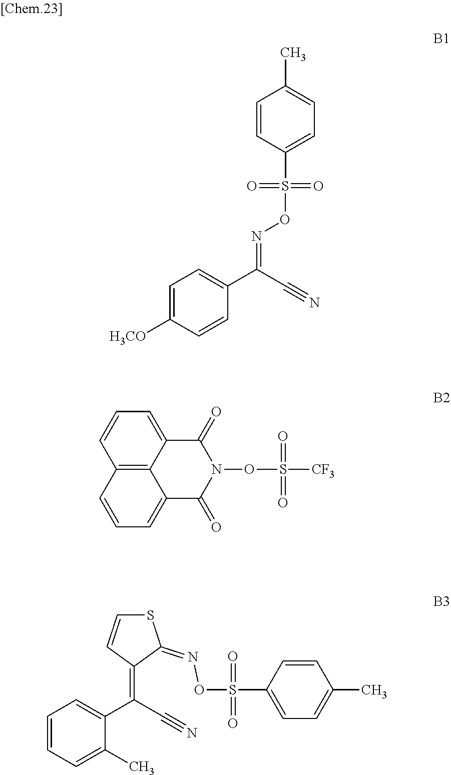 [SMILES: COc1ccc(/C(C#N)=N/OS(=O)(=O)c2ccc(C)cc2)cc1 ; O=C1c2cccc3cccc(c23)C(=O)N1OS(=O)(=O)C(F)(F)F ; Cc1ccc(S(=O)(=O)O/N=C2/SC=C/C2=C(/C#N)c2ccccc2C)cc1]

### 1. Evaluation of Acid Generation Capability

The compositions prepared in Examples and Comparative Examples were each applied to a glass substrate using a spin coater, prebaked at 90° C. for 120 seconds to form a 5-μm thick film, which was post-baked at 250° C. for 3 minutes. The film was developed with a PGMEA solution at 23° C. The acid generation capability of the composition was graded plus (+) when the film remained undissolved or minus (−) when the film did not remain. The results are shown in Table 3. The film's remaining indicates excellent acid generation sensitivity.

### 2. Evaluation of Discoloration

The yellowness index (YI) of the post-baked film formed in the evaluation of acid generation capability was determined. A YI difference (ΔYI) from Comparative Example 1 (YI of the film being evaluated−YI of the comparative film) was calculated and graded in accordance with the following criteria. The results are shown in Table 3.

++: ΔYI is smaller than −1.
+: ΔYI is −1 or greater and smaller than 1.
ΔYI is 1 or greater.
A smaller ΔYI value indicates a higher inhibitory effect on discoloration.

TABLE 3

| | | Example | | | | | | Comparative Example | | | |
|---|---|---|---|---|---|---|---|---|---|---|---|
| | | 7 | 8 | 9 | 10 | 11 | 12 | 1 | 2 | 3 | 4 |
| Epoxy Compound | | 100 | 100 | 100 | 100 | 100 | 100 | 100 | 100 | 100 | 100 |
| Novolak Resin | | 15 | 15 | 15 | 15 | 15 | 15 | 15 | 15 | 15 | 15 |
| Acid | Compound A1 | 1 | | | | | | | | | |
| Generator | Compound A2 | | 1 | | | | | | | | |
| | Compound A3 | | | 1 | | | | | | | |
| | Compound A4 | | | | 1 | | | | | | |
| | Compound A5 | | | | | 1 | | | | | |
| | Compound A6 | | | | | | 1 | | | | |
| | Compound B1 | | | | | | | | 1 | | |
| | Compound B2 | | | | | | | | | 1 | |
| | Compound B3 | | | | | | | | | | 1 |
| Surfactant | | 0.2 | 0.2 | 0.2 | 0.2 | 0.2 | 0.2 | 0.2 | 0.2 | 0.2 | 0.2 |
| Evaluation | Acid | + | + | + | + | + | + | − | + | + | + |
| | Generation | | | | | | | | | | |
| | Capability | | | | | | | | | | |
| | Discoloration | ++ | ++ | ++ | ++ | ++ | ++ | | + | + | − |

### CONCLUSION

The results in Table 3 prove that the compound A exhibits excellent balance between acid generation sensitivity and capability of providing a composition with reduced discoloration.

1. A compound represented by general formula (A): 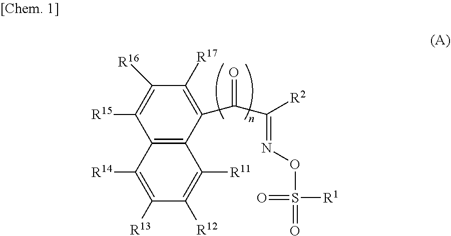
wherein R¹ represents an optionally substituted aliphatic hydrocarbon group having 1 to 20 carbon atoms, an optionally substituted aromatic hydrocarbon group having 6 to 20 carbon atoms, or an optionally substituted heterocyclic group having 2 to 20 carbon atoms, one or more than one methylene group of the aliphatic or aromatic hydrocarbon group or the heterocyclic group being optionally replaced by a divalent group selected from Group I below;
R² represents a hydrogen atom, a halogen atom, a nitro group, a cyano group, an optionally substituted aliphatic hydrocarbon group having 1 to 20 carbon atoms, an optionally substituted aromatic hydrocarbon group having 6 to 20 carbon atoms, or an optionally substituted heterocyclic group having 2 to 20 carbon atoms, one or more than one methylene group of the aliphatic or aromatic hydrocarbon group or the heterocyclic group being optionally replaced by a divalent group selected from Group I below;
R¹¹, R¹², R¹³, R¹⁴, R¹⁵, R¹⁶, and R¹⁷ each independently represent a hydrogen atom, a halogen-atom, a nitro group, a cyano group, a hydroxy group, a carboxy group, R²⁰, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²²;
R²⁰, R²¹, and R²² each independently represent an optionally substituted aliphatic hydrocarbon group having 1 to 20 carbon atoms, an optionally substituted aromatic hydrocarbon group having 6 to 20 carbon atoms, or an optionally substituted heterocyclic group having 2 to 20 carbon atoms, one or more than one methylene group of the aliphatic or aromatic hydrocarbon group or the heterocyclic group being optionally replaced by a divalent group selected from Group I below;
R¹¹ and R¹², R¹² and R¹³, R¹³ and R¹⁴, R¹⁴ and R¹⁵, R¹⁵ and R¹⁶, R¹⁶ and R¹⁷, and R²¹ and R²² may be taken together to form a ring;
a substituent capable of replacing one or more than one hydrogen atom of the aliphatic or aromatic hydrocarbon group or the heterocyclic group being selected from a halogen atom, a cyano group, a nitro group, a hydroxy group, a thiol group, —COOH, —SO₂H, an isocyanate group, and an alkyl group with 1 to 4 carbon atoms; and
n represents 0 or 1.
Group I: —O—, —COO—, —OCO—, —CO—, —CS—, —S—, —SO—, —SO₂—, —NR³⁰—, —NR³⁰—CO—, —CO—NR³⁰—, —NR³⁰—COO—, —OCO—NR³⁰—, and —SiR³⁰R³¹—, wherein R³⁰ and R³¹ each independently represent a hydrogen atom or an unsubstituted aliphatic hydrocarbon group having 1 to 20 carbon atoms.

2. The compound according to claim 1, wherein n is 1.

3. The compound according to claim 1, wherein R¹ is an optionally substituted aliphatic hydrocarbon group having 1 to 20 carbon atoms or an optionally substituted aromatic hydrocarbon group having 6 to 20 carbon atoms.

4. The compound according to claim 3, wherein R¹ is an alkyl group having 1 to 10 carbon atoms with one or more than one of its hydrogen atoms replaced by a halogen atom, an aryl group having 6 to 15 carbon atoms, or an aryl group having 6 to 15 carbon atoms with its hydrogen on the ring replaced by an optionally substituted aliphatic hydrocarbon group.

5. The compound according to claim 1, wherein R¹¹, R¹², R¹³, R¹⁴, R¹⁵, R¹⁶, and R¹⁷ each independently represent a hydrogen atom, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²².

6. The compound according to claim 1, wherein R¹¹, R¹², R¹³, R¹⁴, R¹⁶, and R¹⁷ each represent a hydrogen atom.

7. The compound according to claim 1, wherein R¹⁵ is a hydrogen atom or —OR²⁰.

8. The compound according to claim 1, wherein R² is an alkyl group with 1 to 10 carbon atoms or an aryl group with 6 to 15 carbon atoms.

9. An acid generator comprising the compound according to claim 1.

10. A composition comprising the compound according to claim 1 and a resin component.

11. The composition according to claim 10, wherein the resin component is an acid-reactive component, the acid-reactive component being an acid-curable component or an acid-decomposable component.

12. A cured product obtained from the composition according to claim 11, the acid-reactive component being the acid-curable component.

13. A method for producing a cured product comprising the step of curing the composition according to claim 11, the acid-reactive component being the acid-curable component.

14. A method for forming a patterned coating comprising the steps of:
forming a coating using the composition according to claim 11 and causing the compound present in the coating to generate an acid, and
developing a part of the coating after the acid is generated from the compound,
the acid-reactive component being the acid-decomposable component.

15. The compound according to claim 2, wherein R¹ is an optionally substituted aliphatic hydrocarbon group having 1 to 20 carbon atoms or an optionally substituted aromatic hydrocarbon group having 6 to 20 carbon atoms.

16. The compound according to claim 2, wherein R¹¹, R¹², R¹³, R¹⁴, R¹⁵, R¹⁶, and R¹⁷ each independently represent a hydrogen atom, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²².

17. The compound according to claim 3, wherein R¹¹, R¹², R¹³, R¹⁴, R¹⁵, R¹⁶, and R¹⁷ each independently represent a hydrogen atom, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²².

18. The compound according to claim 4, wherein R¹¹, R¹², R¹³, R¹⁴, R¹⁵, R¹⁶, and R¹⁷ each independently represent a hydrogen atom, —OR²⁰, —COR²⁰, —OCOR²⁰, —COOR²⁰, —SR²⁰, —SOR²⁰, —SO₂R²⁰, —NR²¹R²², —NR²¹COR²², or —CONR²¹R²².

19. The compound according to claim 2, wherein R¹¹, R¹², R¹³, R¹⁴, R¹⁶, and R¹⁷ each represent a hydrogen atom.

20. The compound according to claim 3, wherein R¹¹, R¹², R¹³, R¹⁴, R¹⁶, and R¹⁷ each represent a hydrogen atom.
